(19) United States
(12) Patent Application Publication
(10) Pub. No.: US 2007/0202551 A1
(43) Pub. Date: Aug. 30, 2007

(54) TGFBeta

(76) Inventor: Andrei V. Gudkov, Gates Mills, OH (US)

Correspondence Address: POLSINELLI SHALTON FLANIGAN SUELTHAUS PC, 700 W. 47TH STREET, SUITE 1000, KANSAS CITY, MO, 64112-1802, US

(21) Appl. No.: 11/421,964

(22) Filed: Jun. 2, 2006

Related U.S. Application Data
(63) Continuation of application No. PCT/US04/40656, filed on Dec. 2, 2004.
(60) Provisional application No. 60/526,538, filed on Dec. 2, 2003.
(60) Provisional application No. 60/526,667, filed on Dec. 2, 2003.

(51) Int. Cl. G01N 33/574; C07H 21/04; C12P 21/06; C07K 14/475; A61K 38/18

(52) U.S. Cl. 435/7.23; 435/69.1; 435/320.1; 435/325; 514/12; 530/399; 536/23.5

(57) ABSTRACT

Latent TGFβ induces constitutive activation of NF-κB but does not activate Smad signaling. Latent TGFβ may be used to identify modulators of signaling pathways that are essential for tumor maintenance. Latent TGFβ may also be used to protect a patient from treatments that induce apoptosis.

### CROSS REFERENCE TO RELATED APPLICATIONS

This application is a continuation of International Patent Application No. PCT/US2004/040656, filed Dec. 2, 2006, which claims the benefit of U.S. Provisional Application No. 60/526,538, filed Dec. 2, 2003, and U.S. Provisional Application No. 60/526,667, filed Dec. 2, 2003, the contents of which are incorporated herein by reference.

### FIELD OF THE INVENTION

This invention relates to TGFβ, to methods for using TGFβ in screening assays, and to the use of TGFβ, in particular in the protection of patients from treatments that induce apoptosis.

### BACKGROUND OF THE INVENTION

The progression from normal cells to tumor cells involves a loss of negative mechanisms of growth regulation, including resistance to growth inhibitory stimuli and a lack of dependence on growth factors and hormones. Traditional cancer treatments that are based on radiation or cytotoxic drugs rely on the differences in growth control of normal and malignant cells. Traditional cancer treatments subject cells to severe genotoxic stress. Under these conditions, the majority of normal cells become arrested and therefore saved, while tumor cells continues to divide and die.

However, the nature of conventional cancer treatment strategy is such that normal rapidly dividing or apoptosis-prone tissues are at risk. Damage to these normal rapidly dividing cells causes the well-known side effects of cancer treatment (sensitive tissues: hematopoiesis, small intestine, hair follicles). The natural sensitivity of such tissues is complicated by the fact that cancer cells frequently acquire defects in suicidal (apoptotic) machinery and those therapeutic procedures that cause death in normal sensitive tissues may not be that damaging to cancer cells. Conventional attempts to minimize the side effects of cancer therapies are based on (a) making tumor cells more susceptible to treatment, (b) making cancer therapies more specific for tumor cells, or (c) promoting regeneration of normal tissue after treatment (e.g., erythropoietin, GM-CSF, and KGF).

There continues to be a need for therapeutic agents to mitigate the side effects associated with chemotherapy and radiation therapy in the treatment of cancer. This invention fulfills these needs and provides other related advantages.

### SUMMARY OF THE INVENTION

A modified TGFβ polypeptide is provided. The modification may reduces the rate of conversion of latent TGFβ to active TGFβ. The polypeptide may comprise SEQ ID NO:1, of which R299, R302, or a combination thereof may be substituted with serine. Also provided is a pharmaceutical composition comprising the polypeptide.

Also provided is a method of protecting a patient from a condition that triggers apoptosis. A composition comprising a pharmaceutically effective amount of latent TGFβ may be administered to the patient. The latent TGFβ may be administered prior to, together with, or after a treatment that triggers apoptosis. The treatment may be a cancer treatment. The treatment may also be chemotherapy or radiation therapy. The condition may be a stress, such as radiation, wounding, poisoning, infection or temperature shock.

Also provided is a method of diagnosing a cancer in a mammal. A sample obtained from the mammal may be incubated with an agent that specifically detects the presence of latent TGFβ. Cancer may be diagnosed by comparing the level of binding to a control.

Also provided is a method of screening for a modulator of NF-κB. A suspected modulator and TGFβ may be added to an NF-κB activated expression system. A modulator of NF-κB may be identified by comparing the level of NF-κB activated expression system to a control.

Also provided is a method of screening for a modulator of NF-κB. A suspected modulator and TGFβ may be added to an NF-κB activated expression system. A modulator of NF-κB may be identified by comparing the level of NF-κB activated expression system to a control. The TGFβ may be a latent TGFβ.

Also provided is a method of screening for a modulator of TGFβ. A suspected modulator and TGFβ may be added to a TGFβ activated expression system. A modulator of TGFβ may be identified by comparing the level of TGFβ activated expression system to a control. The TGFβ may be a latent TGFβ.

This invention relates to a method of protecting a patient from one or more treatments or conditions that trigger apoptosis comprising administering to said patient a composition comprising a pharmaceutically effective amount of latent TGFβ. The TGFβ may be administered prior to, together with, or after treatment that triggers apoptosis. The treatment may be a cancer treatment, which may be chemotherapy or radiation therapy. The condition may be a stress, which may be radiation, wounding, poisoning, infection and temperature shock.

This invention also relates to a pharmaceutical composition comprising latent TGFβ and a pharmaceutically acceptable adjuvant, diluent, or carrier. The latent TGFβ may have a modification that reduces the rate of conversion to active TGFβ.

This invention also relates to a method of diagnosis of a cancer in a mammal comprising incubating a sample obtained from said mammal with an agent which specifically detects the presence of latent TGFβ, whereby a cancer is detected by an amount of latent TGFβ higher than in a separate control or reference sample. The agent may be an antibody, which may be a monoclonal antibody.

This invention also relates to a method of screening for a modulator of NF-κB comprising adding a suspected modulator and TGFβ to an NF-κB activated expression system, and separately adding TGFβ to an NF-κB activated expression system, whereby a modulator of NF-κB is identified by the ability to alter NF-κB activated expression. The TGFβ may be latent TGFβ.

This invention also relates to a method of screening for a modulator of TGFβ comprising adding a suspected modulator and TGFβ to a TGFβ activated expression system, and separately adding TGFβ to a TGFβ activated expression system, whereby a modulator of is identified by the ability to alter TGFβ activated expression. The TGFβ may be latent TGFβ.

This invention also relates to a modulator identified by the screening methods described herein. The present invention also relates to a composition comprising a modulator identified by the screening methods described herein. The composition may be a pharmaceutical composition.

### BRIEF DESCRIPTION OF THE DRAWINGS

FIG. 1 demonstrates that cell-free media conditioned from PC3 cells protects normal Balb/c-3T3 fibroblasts from TNF-induced cell death. A: Level of protection from TNF in the presence of CHX for Balb/c-3T3 cells which were previously incubated overnight with medium conditioned by PC3 cells (50, 25 or 12.5%) or Balb/c-3T3 cells (50%). B: Time course of Balb/c-3T3 cells protected by 50% PC3 conditioned medium. C: Activation of NF-κB in Balb/c-3T3 fibroblasts by TNF or by medium conditioned by PC3 cells.

FIG. 2 demonstrates that TGFβ2 secreted by PC3 cells protects Balb/c-3T3 cells from TNF in an NF-κB-dependent manner. A: Protection of Balb/c-3T3 cells from TNF/CHX by conditioned medium from PC3 cells is prevented by anti-TGFβ2 but not by anti-TGFβ1. B: TGFβ2- or PC3-conditioned media protect control cells but not cells transfected with the IκB super-repressor.

FIG. 3 A: Constitutive NF-κB activation in several tumor cell lines. B: Smad2 is not constitutively phosphorylated in the tumor cells. C: Phosphorylation of Smad2 on Ser 465/467 upon TGFβ2 treatment of the tumor cells.

FIG. 4 demonstrates that both the latent and active forms of TGFβ2 activate NF-κB. A: Activation of NF-κB by TGFβ2 in 293 indicator cells. B: Time course of activation of NF-κB by TGFβ2 with IL1-treated cells as controls (note-the time of exposure of the gel for IL1-1 treated cells is much less than for TGFβ2-treated cells). C: TGFβ2-induced IL8 expression in 293 cells. D: NF-κB activation in 293 cells treated with active TGFβs or latent TGFβ1. E: Latent TGFβ1 does not activate Smads. F: Effect of polyclonal anti-TGFβ1 on the activation of NF-κB by latent TGFβ1. G: Activation of NF-κB by latent and active TGFβs in WI38 cells, detected by EMSA.

FIG. 5 demonstrates that inhibition of TGFβ2 production by siRNA suppresses the growth of PC3 cells. A: Reduction in colony number and colony size caused by transduction of siTGFβ2 in PC3 but not in MCF7 cells. The number of colonies per well (average of 3 wells) was determined 10 days after puromycin selection. The experiment was repeated 3 times with similar results. Colony numbers were normalized for transfection efficiency, determined in a β-galactosidase reporter assay (the CMV-LacZ plasmid was added to each transfection mixture). B: PC3 cells stably transfected with a construct expressing siRNA against TGFβ2 express reduced amounts of TGFβ2. The amount of TGFβ2 in culture media conditioned for 24 h by 10⁶ PC3 cells per ml was analyzed by ELISA after activation of TGFβ2 by treatment with 1 M HCl.

FIG. 6 shows the amino acid sequence of the 414 amino acid latent form of TGFβ2 (SEQ ID NO: 1) with the furin cleavage site marked with shading.

FIG. 7 demonstrates expression of TGFβ2 in LNCaP cells transduced with empty lentivurus (V), carrying wild-type TGFβ2 (N) or mutant uncleavable TGFβ2 (R). The samples were run with (+) or without (−) B-mercaptoethanol (ME).

FIG. 8 demonstrates the activation of NF-kB signaling in H1299 luciferase reporter cells by conditioned media from WI-38 cells expressing wild type and uncleavable forms of TGFβ2.

FIG. 9 demonstrates the activation of NF-kB signaling in 293 luciferase reporter cells expressing wild type and uncleavable forms of TGFβ2.

FIG. 10 demonstrates the activation of Smad2 signaling in NIH-3T3 luciferase reporter cells by conditioned media from LNCaP cells expressing wild type and uncleavable forms of TGFβ2 after thermal treatment.

FIG. 11 demonstrates the activation of Smad2 signaling in 293 reporter cells by activated TGFβ2.

FIG. 12 shows adherence and colonies of 293 cells producing normal (N) TGFβ2, uncleavable (R) TGFβ2 or no TGFβ2 after a 24 hours incubation following dilution.

### DETAILED DESCRIPTION

This invention is based on protecting normal cells and tissues from apoptosis caused by stresses including, but not limited to, chemotherapy, radiation therapy and radiation. There are two major mechanisms controlling apoptosis in the cell: the p53 pathway (pro-apoptotic) and the NF-κB pathway (anti-apoptotic). Both pathways are frequently deregulated in tumors: p53 is usually lost, while NF-κB becomes constitutively active. Hence, inhibition of p53 and activation of NF-κB in normal cells may protect them from death caused by stresses, such as cancer treatment, but would not make tumor cells more resistant to treatment because they have these control mechanisms deregulated. This contradicts the conventional view on p53 and NF-κB, which are considered as targets for activation and repression, respectively.

This invention relates to inducing NF-κB activity to protect normal cells from apoptosis. By inducing NF-κB activity in a mammal, normal cells may be protected from apoptosis attributable to cellular stress, which occurs in cancer treatments and hyperthermia; exposure to harmful doses of radiation, for example, workers in nuclear power plants, the defense industry or radiopharmaceutical production, and soldiers; and cell aging. Since NF-κB is constitutively active in many tumor cells, the induction of NF-κB activity may protect normal cells from apoptosis without providing a beneficial effect to tumor cells. Once the normal cells are repaired, NF-κB activity may be restored to normal levels. NF-κB activity may be induced to protect such radiation- and chemotherapy-sensitive tissues as the hematopoietic system (including immune system), the epithelium of the gut, and hair follicles.

Inducers of NF-κB activity may also be used for several other applications. Pathological consequences and death caused by exposure of mammals to a variety of severe conditions including, but not limited to, radiation, wounding, poisoning, infection, aging, and temperature shock, may result from the activity of normal physiological mechanisms of stress response, such as induction of programmed cell death (apoptosis) or release of bioactive proteins, cytokines.

Apoptosis normally functions to “clean” tissues from wounded and genetically damaged cells, while cytokines serve to mobilize the defense system of the organism against the pathogen. However, under conditions of severe injury both stress response mechanisms can by themselves act as causes of death. For example, lethality from radiation may result from massive p53-mediated apoptosis occurring in hematopoietic, immune and digestive systems. Rational pharmacological regulation of NF-κB may increase survival under conditions of severe stress. Control over these factors may allow control of both inflammatory response and the life-death decision of cells from the injured organs.

The protective role of NF-κB is mediated by transcriptional activation of multiple genes coding for: a) anti-apoptotic proteins that block both major apoptotic pathways, b) cytokines and growth factors that induce proliferation and survival of HP and other stem cells, and c) potent ROS-scavenging antioxidant proteins, such as MnSOD (SOD-2). Thus, by temporal activation of NF-κB for radioprotection, it may be possible to achieve not only suppression of apoptosis in cancer patients, but also the ability to reduce the rate of secondary cancer incidence because of simultaneous immunostimulatory effect, which, may be achieved if activation of NF-κB is reached via activation of Toll-like receptors.

Another attractive property of the NF-κB pathway as a target is its activation by numerous natural factors that can be considered as candidate radioprotectants. Among these, are multiple pathogen-associated molecular patterns (PAMPs). PAMPs are molecules that are not found in the host organism, are characteristic for large groups of pathogens, and cannot be easily mutated. They are recognized by Toll-like receptors (TLRs), the key sensor elements of innate immunity. TLRs act as a first warning mechanism of immune system by inducing migration and activation of immune cells directly or through cytokine release. TLRs are type I membrane proteins, known to work as homo-and heterodimers. Upon ligand binding, TLRs recruit MyD88 protein, an indispensable signaling adaptor for most TLRs. The signaling cascade that follows leads to effects including (i) activation of NF-κB pathway, and (ii) activation of MAPKs, including Jun N-terminal kinease (JNK). The activation of the NF-κB pathway by Toll-like receptor ligands makes the ligands attractive as potential radioprotectors. Unlike cytokines, many PAMPs have little effect besides activating TLRs and thus are unlikely to produce side effects. Moreover, many PAMPs are present in humans.

Consistently with their function of immunocyte activation, all TLRs are expressed in spleen and peripheral blood leukocytes, with more TLR-specific patterns of expression in other lymphoid organs and subsets of leukocytes. However, TLRs are also expressed in other tissues and organs of the body, e.g., TLR1 is expressed ubiquitously, TLR5 is also found in GI epithelium and endothelium, while TLRs 2, 6, 7 and 8 are known to be expressed in lung.

1. Definitions

It is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

As used herein, the terms “administer” when used to describe the dosage of an agent that induces NF-κB activity, means a single dose or multiple doses of the agent.

As used herein, the term “analog”, when used in the context of a peptide or polypeptide, means a peptide or polypeptide comprising one or more non-standard amino acids or other structural variations from the conventional set of amino acids.

As used herein, the term “antibody” means an antibody of classes IgG, IgM, IgA, IgD or IgE, or fragments or derivatives thereof, including Fab, F(ab′)₂, Fd, and single chain antibodies, diabodies, bispecific antibodies, bifunctional antibodies and derivatives thereof The antibody may be a monoclonal antibody, polyclonal antibody, affinity purified antibody, or mixtures thereof which exhibits sufficient binding specificity to a desired epitope or a sequence derived therefrom. The antibody may also be a chimeric antibody. The antibody may be derivatized by the attachment of one or more chemical, peptide, or polypeptide moieties known in the art. The antibody may be conjugated with a chemical moiety.

As used herein, “apoptosis” refers to a form of cell death that includes progressive contraction of cell volume with the preservation of the integrity of cytoplasmic organelles; condensation of chromatin (i.e., nuclear condensation), as viewed by light or electron microscopy; and/or DNA cleavage into nucleosome-sized fragments, as determined by centrifuged sedimentation assays. Cell death occurs when the membrane integrity of the cell is lost (e.g., membrane blebbing) with engulfment of intact cell fragments (“apoptotic bodies”) by phagocytic cells.

As used herein, the term “cancer” means any condition characterized by resistance to apoptotic stimuli.

As used herein, the term “cancer treatment” means any treatment for cancer known in the art including, but not limited to, chemotherapy and radiation therapy.

As used herein, the term “combination with” when used to describe administration of an agent that induces NF-κB activity and an additional treatment means that the agent may be administered prior to, together with, or after the additional treatment, or a combination thereof.

As used herein, the term “derivative”, when used in the context of a peptide or polypeptide, means a peptide or polypeptide different other than in primary structure (amino acids and amino acid analogs). By way of illustration, derivatives may differ by being glycosylated, one form of post-translational modification. For example, peptides or polypeptides may exhibit glycosylation patterns due to expression in heterologous systems. If at least one biological activity is retained, then these peptides or polypeptides are derivatives according to the invention. Other derivatives include, but are not limited to, fusion peptides or fusion polypeptides having a covalently modified N- or C-terminus, PEGylated peptides or polypeptides, peptides or polypeptides associated with lipid moieties, alkylated peptides or polypeptides, peptides or polypeptides linked via an amino acid side-chain functional group to other peptides, polypeptides or chemicals, and additional modifications as would be understood in the art.

As used herein, the term “flagellin” means flagellin from any source including, but not limited to, any bacterial species. The flagellin may be from a species of Salmonella. Also specifically contemplated are fragments, variants, analogs, homologs, or derivatives of said flagellin, and combinations thereof. The various fragments, variants, analogs, homologs or derivatives described herein may be 50%, 55%, 60%, 65%, 70%, 75%, 80%, 85%, 90%, 95%, 97%, 98%, or 99% identical to a wild-type flagellin.

As used herein, the term “fragment”, when used in the context of a peptide or polypeptide, means a peptides of from about 8 to about 50 amino acids in length. The fragment may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length.

As used herein, the term “homolog”, when used in the context of a peptide or polypeptide, means a peptide or polypeptide sharing a common evolutionary ancestor.

As used herein, the term “latent TGFβ” means a precursor of TGFβ that is not in an active form. A latent TGFβ may be a precursor of TGFβ containing active TGFβ and latency-associated peptide (LAP). A latent TGFβ may also comprise LAP linked to latent TGFβ binding protein. A latent TGFβ may also be the large latent complex. Furthermore, a latent TGFβ may be a latent TGFβ that is modified so that the rate of conversion to active TGFβ or ability to be converted to TGFβ has been reduced. The modified latent TGFβ may be, for example, a TGFβ mutant that prevents or reduces conversion to active TGFβ.

As used herein, the term “TGFβ” means any isoform of active or latent TGFβ including, but not limited to, TGFβ1, TGFβ2, TGFβ3, TGFβ4 or TGFβ5, and combinations thereof. Also specifically contemplated are fragments, variants, analogs, homologs, or derivatives of said TGFβ isoforms, and combinations thereof. The various fragments, variants, analogs, homologs or derivatives described herein may be 50%, 55%, 60%, 65%, 70%, 75%, 80%, 85%, 90%, 95%, 97%, 98%, or 99% identical to a TGFβ isoform.

As used herein, the term “treat” or “treating” when referring to protection of a mammal from a condition, means preventing, suppressing, repressing, or eliminating the condition. Preventing the condition involves administering a composition of this invention to a mammal prior to onset of the condition. Suppressing the condition involves administering a composition of this invention to a mammal after induction of the condition but before its clinical appearance. Repressing the condition involves administering a composition of this invention to a mammal after clinical appearance of the condition such that the condition is reduced or maintained. Elimination the condition involves administering a composition of this invention to a mammal after clinical appearance of the condition such that the mammal no longer suffers the condition.

As used herein, the term “tumor cell” means any cell characterized by resistance to apoptotic stimuli.

As used herein, the term “variant”, when used in the context of a peptide or polypeptide, means a peptide or polypeptide that differs in amino acid sequence by the insertion, deletion, or conservative substitution of amino acids, but retain at least one biological activity. For purposes of this invention, “biological activity” includes, but is not limited to, the ability to be bound by a specific antibody. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ∀ 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated herein by reference. Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

2. Methods of Treatment

a. Constitutively Active NF-κB Tumor

This invention relates to a method of treating a mammal suffering from a constitutively active NF-κB cancer comprising administering to the mammal a composition comprising a therapeutically effective amount of an agent that induces NF-κB activity. The agent that induces NF-κB activity may be administered in combination with a cancer treatment.

The agent may be administered simultaneously or metronomically with other anti-cancer treatments such as chemotherapy and radiation therapy. The term “simultaneous” or “simultaneously” as used herein, means that the other anti-cancer treatment and the compound of the present invention administered within 48 hours, preferably 24 hours, more preferably 12 hours, yet more preferably 6 hours, and most preferably 3 hours or less, of each other. The term “metronomically” as used herein means the administration of the compounds at times different from the chemotherapy and at certain frequency relative to repeat administration and/or the chemotherapy regiment.

The agent may be administered at any point prior to exposure to the cancer treatment including, but not limited to, about 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 3 hr, 2 hr, or 1 hr prior to exposure. The agent may be administered at any point after exposure to the cancer treatment including, but not limited to, about 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, or 48 hr after exposure.

The cancer treatment may comprise administration of a cytotoxic agent or cytostatic agent, or combination thereof. Cytotoxic agents prevent cancer cells from multiplying by: (1) interfering with the cell's ability to replicate DNA and (2) inducing cell death and/or apoptosis in the cancer cells. Cytostatic agents act via modulating, interfering or inhibiting the processes of cellular signal transduction which regulate cell proliferation and sometimes at low continuous levels.

Classes of compounds that may be used as cytotoxic agents include the following: alkylating agents (including, without limitation, nitrogen mustards, ethylenimine derivatives, alkyl sulfonates, nitrosoureas and triazenes): uracil mustard, chlormethine, cyclophosphamide (Cytoxan®), ifosfamide, melphalan, chlorambucil, pipobroman, triethylene-melamine, triethylenethiophosphoramine, busulfan, carmustine, lomustine, streptozocin, dacarbazine, and temozolomide; antimetabolites (including, without limitation, folic acid antagonists, pyrimidine analogs, purine analogs and adenosine deaminase inhibitors): methotrexate, 5-fluorouracil, floxuridine, cytarabine, 6-mercaptopurine, 6-thioguanine, fludarabine phosphate, pentostatine, and gemcitabine; natural products and their derivatives (for example, vinca alkaloids, antitumor antibiotics, enzymes, lymphokines and epipodophyllotoxins): vinblastine, vincristine, vindesine, blcomycin, dactinomycin, daunorubicin, doxorubicin, epirubicin, idarubicin, ara-c, paclitaxel (paclitaxel is commercially available as Taxol®), mithramycin, deoxyco-formycin, mitomycin-c, 1-asparaginase, interferons (preferably IFN-α), etoposide, and teniposide.

Other proliferative cytotoxic agents are navelbene, CPT-11, anastrazole, letrazole, capecitabine, reloxafine, cyclophosphamide, ifosamide, and droloxafine.

Microtubule affecting agents interfere with cellular mitosis and are well known in the art for their cytotoxic activity. Microtubule affecting agents useful in the invention include, but are not limited to, allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, and discodermolide (see Service, (1996) Science, 274:2009) estramustine, nocodazole, MAP4, and the like. Examples of such agents are also described in Bulinski (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; and Panda (1996) J. Biol. Chem 271:29807-29812.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Cytostatic agents that may be used include, but are not limited to, hormones and steroids (including synthetic analogs): 17.alpha.-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, hlorotrianisene, hydroxyprogesterone, aminoglutethimide, estramustine, medroxyprogesteroneacetate, leuprolide, flutamide, toremifene, zoladex.

Other cytostatic agents are antiangiogenics such as matrix metalloproteinase inhibitors, and other VEGF inhibitors, such as anti-VEGF antibodies and small molecules such as ZD6474 and SU6668 are also included. Anti-Her2 antibodies from Genetech may also be utilized. A suitable EGFR inhibitor is EKB-569 (an irreversible inhibitor). Also included are Imclone antibody C225 immunospecific for the EGFR, and src inhibitors.

Also suitable for use as an cytostatic agent is Casodex® (bicalutamide, Astra Zeneca) which renders androgen-dependent carcinomas non-proliferative. Yet another example of a cytostatic agent is the antiestrogen Tamoxifen® which inhibits the proliferation or growth of estrogen dependent breast cancer. Inhibitors of the transduction of cellular proliferative signals are cytostatic agents. Representative examples include epidermal growth factor inhibitors, Her-2 inhibitors, MEK-1 kinase inhibitors, MAPK kinase inhibitors, PI3 inhibitors, Src kinase inhibitors, and PDGF inhibitors.

A variety of cancers may be treated according to this invention including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, and teratocarcinoma. In a preferred embodiment, this invention is used to treat cancers of gastrointestinal tract.

b. Treatment of Side Effects from Cancer Treatment

This invention also relates to a method of treating a mammal suffering from damage to normal tissue attributable to treatment of a constitutively active NF-κB cancer, comprising administering to the mammal a composition comprising a therapeutically effective amount of an agent that induces NF-κB activity. The agent that induces NF-κB activity may be administered in combination with a cancer treatment described above.

c. Modulation of Cell Aging

This invention also relates to a method of modulating cell aging in a mammal, comprising administering to the mammal a therapeutically effective amount of an agent that induces NF-κB activity. The agent that induces NF-κB activity may be administered in combination with other treatments.

The agent may be administered at any point prior to administration of the other treatment including, but not limited to, about 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, or 1 hr prior to administration. The agent may be administered at any point after administration of the other treatment including, but not limited to, about 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, or 48 hr after administration.

d. Treatment of Stress

This invention also relates to a method of treating a mammal suffering from damage to normal tissue attributable to stress, comprising administering to the mammal a composition comprising a therapeutically effective amount of an agent that induces NF-κB activity. The agent that induces NF-κB activity may be administered in combination with other treatments. The stress may be attributable to any source including, but not limited to, radiation, wounding, poisoning, infection, and temperature shock.

The composition comprising an inducer of NF-κB may be administered at any point prior to exposure to the stress including, but not limited to, about 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, or 1 hr prior to exposure. The composition comprising an inducer of NF-κB may be administered at any point after exposure to the stress including, but not limited to, about 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, or 48 hr after exposure.

e. Radiation

This invention is also related to the protection of cells from the effects of exposure to radiation. Injury and death of normal cells from ionizing radiation is a combination of a direct radiation-induced damage to the exposed cells and an active genetically programmed cell reaction to radiation-induced stress resulting in a suicidal death or apoptosis. Apoptosis plays a key role in massive cell loss occurring in several radiosensitive organs (i.e., hematopoietic and immune systems, epithelium of digestive tract, etc.), the failure of which determines general radiosensitivity of the organism.

Exposure to ionizing radiation (IR) may be short- or long-term, it may be applied as a single or multiple doses, to the whole body or locally. Thus, nuclear accidents or military attacks may involve exposure to a single high dose of whole body irradiation (sometimes followed by a long-term poisoning with radioactive isotopes). The same is true (with strict control of the applied dose) for pretreatment of patients for bone marrow transplantation when it is necessary to prepare hematopoietic organs for donor's bone marrow by “cleaning” them from the host blood precursors. Cancer treatment may involve multiple doses of local irradiation that greatly exceeds lethal dose if it were applied as a total body irradiation. Poisoning or treatment with radioactive isotopes results in a long-term local exposure to radiation of targeted organs (e.g., thyroid gland in the case of inhalation of 125I). Finally, there are many physical forms of ionizing radiation differing significantly in the severity of biological effects.

At the molecular and cellular level, radiation particles are able to produce breakage and cross-linking in the DNA, proteins, cell membranes and other macromolecular structures. Ionizing radiation also induces the secondary damage to the cellular components by giving rise to the free radicals and reactive oxygen species (ROS). Multiple repair systems counteract this damage, such as several DNA repair pathways that restore the integrity and fidelity of the DNA, and antioxidant chemicals and enzymes that scavenge the free radicals and ROS and reduce the oxidized proteins and lipids. Cellular checkpoint systems detect the DNA defects and delay cell cycle progression until damage is repaired or decision to commit cell to growth arrest or programmed cell death (apoptosis) is reached

Radiation can cause damage to mammalian organism ranging from mild mutagenic and carcinogenic effects of low doses to almost instant killing by high doses. Overall radiosensitivity of the organism is determined by pathological alterations developed in several sensitive tissues that include hematopoietic system, reproductive system and different epithelia with high rate of cell turnover.

Acute pathological outcome of gamma irradiation leading to death is different for different doses and is determined by the failure of certain organs that define the threshold of organism's sensitivity to each particular dose. Thus, lethality at lower doses occurs from bone marrow aplasia, while moderate doses kill faster by inducing a gastrointestinal (GI) syndrome. Very high doses of radiation can cause almost instant death eliciting neuronal degeneration.

Organisms that survive a period of acute toxicity of radiation can suffer from long-term remote consequences that include radiation-induced carcinogenesis and fibrosis developing in exposed organs (e.g., kidney, liver or lungs) months and years after irradiation.

Cellular DNA is the major target of IR that causes a variety of types of DNA damage (genotoxic stress) by direct and indirect (free radical-based) mechanisms. All organisms maintain DNA repair system capable of effective recovery of radiation-damaged DNA; errors in DNA repair process may lead to mutations.

Tumors are generally more sensitive to gamma radiation and can be treated with multiple local doses that cause relatively low damage to normal tissue. Nevertheless, in some instances, damage of normal tissues is a limiting factor in application of gamma radiation for cancer treatment. The use of gamma-irradiation during cancer therapy by conventional, three-dimensional conformal or even more focused BeamCath delivery has also dose-limiting toxicities caused by cumulative effect of irradiation and inducing the damage of the stem cells of rapidly renewing normal tissues, such as bone marrow and gastrointestinal (GI) tract.

At high doses, radiation-induced lethality is associated with so-called hematopoietic and gastrointestinal radiation syndromes. Hematopoietic syndrome is characterized by loss of hematopoietic cells and their progenitors making it impossible to regenerate blood and lymphoid system. The death usually occurs as a consequence of infection (result of immunosuppression), hemorrhage and/or anemia. GI syndrome is caused by massive cell death in the intestinal epithelium, predominantly in the small intestine, followed by disintegration of intestinal wall and death from bacteriemia and sepsis. Hematopoietic syndrome usually prevails at the lower doses of radiation and leads to the more delayed death than GI syndrome.

In the past, radioprotectants were typically antioxidants—both synthetic and natural. More recently, cytokines and growth factors have been added to the list of radioprotectants; the mechanism of their radioprotection is considered to be a result of facilitating effect on regeneration of sensitive tissues. There is no clear functional distinction between both groups of radioprotectants, however, since some cytokines induce the expression of the cellular antioxidant proteins, such as manganese superoxide dismutase (MnSOD) and metallothionein.

The measure of protection for a particular agent is expressed by dose modification factor (DMF or DRF). DMF is determined by irradiating the radioprotector treated subject and untreated control subjects with a range of radiation doses and then comparing the survival or some other endpoints. DMF is commonly calculated for 30-day survival (LD50/30 drug-treated divided by LD50/30 vehicle-treated) and quantifies the protection of the hematopoietic system. In order to estimate gastrointestinal system protection, LD50 and DMF are calculated for 6- or 7-day survival. DMF values provided herein are 30-day unless indicated otherwise.

Inducers of NF-κB possess strong pro-survival activity at the cellular level and on the organism as a whole. In response to super-lethal doses of radiation, inducers of NF-κB inhibit both gastrointestinal and hematopoietic syndromes, which are the major causes of death from acute radiation exposure. As a result of these properties, inducers of NF-κB may be used to treat the effects of natural radiation events and nuclear accidents. Moreover, since inducers of NF-κB acts through mechanisms different from all presently known radioprotectants, they can be used in combination with other radioprotectants, thereby, dramatically increasing the scale of protection from ionizing radiation.

As opposed to conventional radioprotective agents (e.g., scavengers of free radicals), anti-apoptotic agents may not reduce primary radiation-mediated damage but may act against secondary events involving active cell reaction on primary damage, therefore complementing the existing lines of defense. Pifithrin-alpha, a pharmacological inhibitor of p53 (a key mediator of radiation response in mammalian cells), is an example of this new class of radioprotectants. However, the activity of p53 inhibitors is limited to protection of the hematopoietic system and has no protective effect in digestive tract (gastrointestinal syndrome), therefore, reducing therapeutic value of these compounds. Anti-apoptotic pharmaceuticals with broader range of activity are desperately needed.

Inducers of NF-κB may be used as a radioprotective agent to extend the range of tolerable radiation doses by increasing radioresistance of human organism beyond the levels achievable by currently available measures (shielding and application of existing bioprotective agents) and drastically increase the chances of crew survival in case of onboard nuclear accidents or large-scale solar particle events. With an approximate DMF (30-day survival) greater than 1.5, the NF-κB inducer flagellin is more effective than any currently reported natural compound.

Inducers of NF-κB are also useful for treating irreplaceable cell loss caused by low-dose irradiation, for example, in the central nervous system and reproductive organs. Inducers of NF-κB may also be used during cancer chemotherapy to treat the side effects associated with chemotherapy, including alopecia.

In one embodiment, a mammal is treated for exposure to radiation, comprising administering to the mammal a composition comprising a therapeutically effective amount of a composition comprising an inducer of NF-κB. The composition comprising an inducer of NF-κB may be administered in combination with one or more radioprotectants. The one or more radioprotectants may be any agent that treats the effects of radiation exposure including, but not limited to, antioxidants, free radical scavengers and cytokines.

Inducers of NF-κB may inhibit radiation-induced programmed cell death in response to damage in DNA and other cellular structures; however, inducers of NF-κB may not deal with damage at the cellular and may not prevent mutations. Free radicals and reactive oxygen species (ROS) are the major cause of mutations and other intracellular damage. Antioxidants and free radical scavengers are effective at preventing damage by free radicals. The combination of an inducer of NF-κB and an antioxidant or free radical scavenger may result in less extensive injury, higher survival, and improved health for mammal exposed to radiation. Antioxidants and free radical scavengers that may be used in the practice of the invention include, but are not limited to, thiols, such as cysteine, cysteamine, glutathione and bilirubin; amifostine (WR-2721); vitamin A; vitamin C; vitamin E; and flavonoids such as Indian holy basil (Ocimum sanctum), orientin and vicenin.

Inducers of NF-κB may also be administered in combination with a number of cytokines and growth factors that confer radioprotection by replenishing and/or protecting the radiosensitive stem cell populations. Radioprotection with minimal side effects may be achieved by the use of stem cell factor (SCF, c-kit ligand), Flt-3 ligand, and interleukin-1 fragment IL-1b-rd. Protection may be achieved through induction of proliferation of stem cells (all mentioned cytokines), and prevention of their apoptosis (SCF). The treatment allows accumulation of leukocytes and their precursors prior to irradiation thus enabling quicker reconstitution of the immune system after irradiation. SCF efficiently rescues lethally irradiated mice with DMF in range 1.3-1.35 and is also effective against gastrointestinal syndrome. Flt-3 ligand also provides strong protection in mice (70-80% 30-day survival at LD100/30, equivalent to DMF>1.2) and rabbits (15, 16).

Several factors, while not cytokines by nature, stimulate the proliferation of the immunocytes and may be used in combination with inducers of NF-κB. 5-AED (5-androstenediol) is a steroid that stimulates the expression of cytokines and increases resistance to bacterial and viral infections. A subcutaneous injection of 5-AED in mice 24 h before irradiation improved survival with DMF=1.26. Synthetic compounds, such as ammonium tri-chloro(dioxoethyle-O,O′-)tellurate (AS-101), may also be used to induce secretion of numerous cytokines and for combination with inducers of NF-κB.

Growth factors and cytokines may also be used to provide protection against the gastrointestinal syndrome. Keratinocyte growth factor (KGF) promotes proliferation and differentiation in the intestinal mucosa, and increases the post-irradiation cell survival in the intestinal crypts. Hematopoietic cytokine and radioprotectant SCF may also increase intestinal stem cell survival and associated short-term organism survival.

Inducers of NF-κB may offer protection against both gastrointestinal (GI) and hematopoietic syndromes. Since mice exposed to 15 Gy of whole-body lethal irradiation died mostly from GI syndrome, a composition comprising an inducer of NF-κB and one or more inhibitors of GI syndrome may be more effective. Inhibitors of GI syndrome that may be used in the practice of the invention include, but are not limited to, cytokines such as SCF and KGF.

The composition comprising an inducer of NF-κB may be administered at any point prior to exposure to radiation including, but not limited to, about 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, or 1 hr prior to exposure. The composition comprising an inducer of NF-κB may be administered at any point after exposure to radiation including, but not limited to, about 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, or 48 hr after exposure to radiation.

3. Agent

This invention also relates to an agent that induces NF-κB activity. The agent may be an artificially synthesized compound or a naturally occurring compound. The agent may be a low molecular weight compound, polypeptide or peptide, or a fragment, analog, homolog, variant or derivative thereof.

The agent may also be an NF-κB inducing cytokine including, but not limited to, IL2, IL6, TNF and TGFβ. The agent may also be a prostaglandin. The agent may also be a growth factor including, but not limited to, KGF and PDGF. The agent may also be an antibody that induces NF-κB activity.

a. TGFβ

In one embodiment, the NF-κB inducing agent is TGFβ. As shown in the Examples below, latent TGFβ2, which is secreted by many tumor cells is required for their continued survival and proliferation through the mechanism of NF-κB activation. Importantly, latent TGFβ2 does not activate Smad signaling, thus separating its pro-survival effects from the growth inhibiting and immunosuppressive functions of active TGFβ2. Bacterial flagellin, which is a potent inductor of NF-κB, has recently been shown to be a potent radioprotector. Similarly, the latent form of TGFβ2 may also be used as a radioprotector through its activation of NF-κB.

Three mammalian TGFβ isoforms, TGFβ1, TGFβ2, and TGFβ3, have been identified. In general, they exhibit similar functions in vitro, most notably on cell growth regulation, ECM production, and immune modulation. The TGFβs bind to a heteromeric complex of transmembrane serine/threonine kinases, the type I and type II receptors (TβRI and TβRII). All TGFβs are secreted as latent precursors containing active TGFβ moiety linked to a latency-associated peptide (LAP). In most cells, LAP is bound to an additional protein, latent TGFβ binding protein, forming a large latent complex. Latent TGFβs must be processed to mature forms (to release the mature TGFβ peptide) in order to activate the receptors that mediate Smad-dependent signaling. The activation of TGFβ is a complex process involving conformational changes of latent TGFβ, induced either by cleavage of LAP by proteases, the actions of endoglycosylases or by the binding of LAP to proteins such as integrin αvβ5 or thrombospondin-1.

Functional interaction between TGFβ and NF-κB in tumor cells has been addressed in several previous publications. However, these studies have provided a controversial picture, in which TGFβ acts as either an inhibitor or activator of NF-κB-mediated signaling. Importantly, this activation does not go through classical TGFβ intracellular signaling (involving Smad2 and 3), but through an alternative signaling pathway that involves TGFβ-activated kinase 1 (TAK1), a member of the MAPK family. Activation of TAK1 leads to the phosphorylation of IκB kinase, which, in turn, leads to the phosphorylation and subsequent degradation of IκB and activation of NF-κB. Secretion of TGFβ causes a dual effect on tumor cells of epithelial origin. On the one hand, it is growth suppressive as a result of Smad-dependent signaling. On the other hand, TGFβ secretion can promote tumor cell survival by the permanent activation of NF-κB through the TAK1-IκB kinase pathway.

Our findings, together with what is already known about the functional interactions between TGFβ and NF-κB, shows that secretion of TGFβ causes a dual effect on the cells of epithelial origin. On the one hand, activated TGFβ2 is growth suppressive for these cells due to Smad-dependent signaling. On the other hand, TGFβ secretion can be beneficial for these cells by the activation of the NF-κB-mediated survival through the TAK1-IκB kinase pathway. The use of recombinant latent TGFβ2 for the prevention of cell death from γ-irradiation via NF-κB activation has certain advantages over some chemical drugs and other cytokines. First, latent TGFβ2 is normally present in blood and tissues in the small amounts that usually do not induce any pathological effect. Second, this ˜80 kDa protein may not be immunogenic and therefore may be used repeatedly. Furthermore, it may not induce immunosuppression, unlike active TGFβ2, which usually activates the Smad-dependent inhibitory pathway. In addition, the problem of the processing of latent TGFβ2 into its active form through protease cleavage of the LAP portion may be resolved by the creation of a non-cleavable mutant of latent TGFβ2.

A fragment, variant, analog, homolog, or derivative of an inducer of NF-κB, such as TGFβ, with beneficial properties may be obtained by rational-based design based on the domain structure of TGFβ. One one embodiment, the inducer of NF-κB is uncleavable form of latent TGFβ2 (uL-TGFβ2). In a preferred embodiment, the agent is a polypeptide comprising SEQ ID NO:1, wherein R299 or R302 is substituted with another amino acid, such as serine. In another preferred embodiment, the agent is a polypeptide comprising SEQ ID NO:1, wherein R299 and R302 are substituted with another amino acid, such as serine.

4. Composition

This invention also relates to a composition comprising a therapeutically effective amount of an inducer of NF-κB. The composition may be a pharmaceutical composition, which may be produced using methods well known in the art. As described above, the composition comprising an inducer of NF-κB may be administered to a mammal for the treatment of conditions associated with apoptosis including, but not limited to, exposure to radiation, side effect from cancer treatments, stress and cell aging. The composition may also comprise additional agents including, but not limited to, a radioprotectant or a chemotherapeutic drug.

a. Administration

Compositions of this invention may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. For veterinary use, the composition may be administered as a suitably acceptable formulation in accordance with normal veterinary practice. The veterinarian can readily determine the dosing regimen and route of administration that is most appropriate for a particular animal.

b. Formulation

Compositions of this invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

Compositions of this invention may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

Compositions of this invention may also be formulated as suppositories, which may contain suppository bases including, but not limited to, cocoa butter or glycerides. Compositions of this invention may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. Compositions of this invention may also be formulated transdermal formulations comprising aqueous or nonaqueous vehicles including, but not limited to, creams, ointments, lotions, pastes, medicated plaster, patch, or membrane.

Compositions of this invention may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions of this invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

c. Dosage

A therapeutically effective amount of the agent required for use in therapy varies with the nature of the condition being treated, the length of time that induction of NF-κB activity is desired, and the age and the condition of the patient, and is ultimately determined by the attendant physician. In general, however, doses employed for adult human treatment typically are in the range of 0.001 mg/kg to about 200 mg/kg per day. The dose may be about 1 μg/kg to about 100 μg/kg per day. The desired dose may be conveniently administered in a single dose, or as multiple doses administered at appropriate intervals, for example as two, three, four or more subdoses per day. Multiple doses often are desired, or required, because NF-κB activity in normal cells may be decreased once the agent is no longer administered.

The dosage of an inducer of NF-κB may be at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg or 1 mg/kg.

5. Screening Methods

This invention also relates to methods of identifying agents that induce NF-κB activity. An agent that induces NF-κB activity may be identified by a method comprising adding a suspected inducer of NF-κB activity to an NF-κB activated expression system, comparing the level of NF-κB activated expression to a control, whereby an inducer of NF-κB activity is identified by the ability to increase the level of NF-κB activated expression system.

Candidate agents may be present within a library (i.e., a collection of compounds). Such agents may, for example, be encoded by DNA molecules within an expression library. Candidate agent be present in conditioned media or in cell extracts. Other such agents include compounds known in the art as “small molecules,” which have molecular weights less than 10⁵ daltons, preferably less than 10⁴ daltons and still more preferably less than 10³ daltons. Such candidate agents may be provided as members of a combinatorial library, which includes synthetic agents (e.g., peptides) prepared according to multiple predetermined chemical reactions. Those having ordinary skill in the art will appreciate that a diverse assortment of such libraries may be prepared according to established procedures, and members of a library of candidate agents can be simultaneously or sequentially screened as described herein.

The screening methods may be performed in a variety of formats, including in vitro, cell-based and in vivo assays. Any cells may be used with cell-based assays. Preferably, cells for use with this invention include mammalian cells, more preferably human and non-human primate cells. Cell-base screening may be performed using genetically modified tumor cells expressing surrogate markers for activation of NF-κB. Such markers include, but are not limited to, bacterial beta-galactosidase, luciferase and enhanced green fluorescent protein (EGFP). The amount of expression of the surrogate marker may be measured using techniques standard in the art including, but not limited to, colorimetery, luminometery and fluorimetery.

The conditions under which a suspected modulator is added to a cell, such as by mixing, are conditions in which the cell can undergo apoptosis or signaling if essentially no other regulatory compounds are present that would interfere with apoptosis or signaling. Effective conditions include, but are not limited to, appropriate medium, temperature, pH and oxygen conditions that permit cell growth. An appropriate medium is typically a solid or liquid medium comprising growth factors and assimilable carbon, nitrogen and phosphate sources, as well as appropriate salts, minerals, metals and other nutrients, such as vitamins, and includes an effective medium in which the cell can be cultured such that the cell can exhibit apoptosis or signaling. For example, for a mammalian cell, the media may comprise Dulbecco's modified Eagle's medium containing 10% fetal calf serum.

Cells may be cultured in a variety of containers including, but not limited to tissue culture flasks, test tubes, microtiter dishes, and petri plates. Culturing is carried out at a temperature, pH and carbon dioxide content appropriate for the cell. Such culturing conditions are also within the skill in the art.

Methods for adding a suspected modulator to the cell include electroporation, microinjection, cellular expression (i.e., using an expression system including naked nucleic acid molecules, recombinant virus, retrovirus expression vectors and adenovirus expression), use of ion pairing agents and use of detergents for cell permeabilization.

6. Diagnostic

The present invention also relates to a diagnostic for detecting the presence of TGFβ. The diagnostic may be specific for latent TGFβ. The diagnostic may be an antibody specific for latent TGFβ.

a. Use of Diagnostic

A diagnostic for latent TGFβ may be used for the diagnosis of a cancer. A diagnostic for latent TGFβ may also be used to diagnose a risk of cancer. A diagnostic for latent TGFβ may also be used to monitor the progression of a cancer. A diagnostic for latent TGFβ may also be used to monitor the effect of treatment in a cancer patient.

7. Antibodies

The present invention is also related to an antibody that specifically binds to latent TGFβ. The antibody may be used as a diagnostic as described above. The antibody may also be used as a treatment to lower levels of latent TGFβ. The antibody may also be used to limit constitutive activation of NF-κB. The antibody may delivered by a variety of administrative routes, in pharmaceutical compositions comprising carriers or diluents, as would be understood by one of skill in the art.

The antibody may be produced by using standard techniques, including as described in WO 01/55210, the contents of which are hereby incorporated by reference in their entirety. The antibody may also be any commercially available antibody that is specific for latent TGFβ.

The antibodies of the present invention include antibodies of classes IgG, IgM, IgA, IgD, and IgE, and fragments and derivatives thereof including Fab and F(ab′)₂. The antibodies may also be recombinant antibody products including, but not limited to, single chain antibodies, chimeric antibody products, “humanized” antibody products, and CDR-grafted antibody products. The antibodies of the present invention include monoclonal antibodies, polyclonal antibodies, affinity purified antibodies, or mixtures thereof which exhibit sufficient binding specificity to latent TGFβ. The antibody may also be an antibody fragment.

The antibody many also be attached to a label. Labels can be signal-generating enzymes, antigens, other antibodies, lectins, carbohydrates, biotin, avidin, radioisotopes, toxins, heavy metals, and other compositions known in the art. Attachment techniques are also well known in the art.

This invention has multiple aspects, illustrated by the following non-limiting examples.

### EXAMPLE 1

### TNF-Resistant Prostate Cancer Cells Protect TNF-Sensitive Cells From Apoptosis

The human prostate tumor cell lines PC3 and DU145 are consistently resistant to treatment with TNF, while mouse fibroblast Balb/c-3T3 indicator cells are highly sensitive to TNF in the presence of low levels of cyclohexamide (CHX) (Gasparian et al., 2002). Balb/c-3T3 cells were incubated overnight, separately, or together at a ratio of 5:1:: Balb/c-3T3 :PC3 in RPMI-1640 medium with 10% FCS, followed by the addition of CHX or a combination of CHX (0.4 μg/ml, Sigma, St. Louis, Mo.) and TNFα (0.2 ng/ml, PeproTech Inc., Rocky Hill, N.J.). As a control, Balb/c-3T3 cells were also incubated with the human prostate tumor cell line LNCaP, which are sensitive to TNF (Palayoor et al., 1999). Balb/c-3T3 cells cultivated with PC3 cells were protected from TNF-induced apoptosis, whereas LNCaP do not provide protection (data not shown).

### EXAMPLE 2

### Media Conditioned by TNF-Resistant Prostate Cancer Cells Protect TNF-Sensitive Cells from Apoptosis

To determine whether the resistance to TNF-induced apoptosis provided by PC3 cells in Example 1 was an intrinsic or transmissible trait, cell-free media conditioned by PC3 cells or LNCaP cells was tested for the ability to protect Balb/c-3T3 cells from TNF-induced apoptosis in the presence of CHX. To collect conditioned media, equal number of cells were cultivated to ˜90% confluency, the media was replaced, and the cells were incubated for another 24 h. Samples of conditioned media were filtered, aliquotted, and frozen at −70° C. for future use.

Conditioned medium from PC3 or LNCaP cells (50, 25 or 12.5%) was used to treat Balb/c-3T3 cells overnight before addition of TNF and CHX. Unconditioned medium was used as a control. Cells were counted by first washing with 1× phosphate buffered saline (PBS) followed by staining with methylene blue, solubilization with 30% HCl, and measuring absorbance at 640 nm.

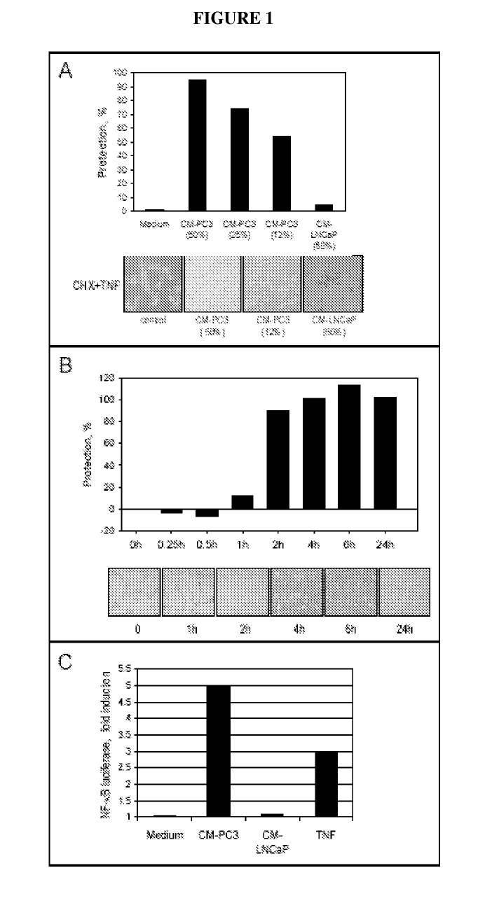

FIGS. 1A and 1B indicates that overnight treatment with cell-free media conditioned by PC3 cells, but not LNCaP cells, protects Balb/c-3T3 cells from apoptosis mediated by TNF plus CHX in a dose- and time-dependent manner. The anti-TNF effect did not require the continued presence of conditioned medium (data not shown), suggesting that the mechanism is unlikely to involve the direct inactivation of TNF, but rather to require the induction of TNF resistance in the indicator cells, which manifest approximately 2 h after pretreatment (FIG. 1B).

### EXAMPLE 3

### Media Conditioned by TNF-Resistant Prostate Cancer Cells Induce NF-κB

The TNF-resistance of PC3 and many other types of cells is mediated by the activity of NF-κB (Muenchen et al., 2000). The DNA-binding activity of NF-κB is constitutively high in the human prostate tumor cell lines PC3 and DU145, but not the human prostate tumor cell line LNCaP, (Gasparian et al., 2002; Palayoor et al., 1999). Therefore, we tested whether the culture media conditioned by PC3 cells in Example 1 was able to induce NF-κB activity in addition to protecting cells from apoptosis.

Balb/c-3T3 cells were transiently transfected with an NF-κB reporter construct. Efficiencies of transient transfection were normalized by determining β-galactosidase activity in cells co-transfected with a pCMVLacZ β-galactosidase reporter plasmid. Twenty-four hours after transfection, media conditioned by PC3 or LNCaP cells was added. Medium and TNFα were also individually added as negative and positive controls, respectively. After an additional 24 hours, NF-κB activity was determine by assaying luciferase activity following the protocol provided by Promega Corporation, Madison, Wis. FIG. 1C shows that NF-κB activity is induced by media conditioned by PC3 cells, but not LNCaP cells.

### EXAMPLE 4

### TGFβ2 Mediates the Anti-Apoptotic Effect of PC3-Conditioned Media

Based on the results in Example 2, we were interested in whether we could identity the factors produced by PC3 cells that inhibit TNF-mediated apoptosis. Among the many cytokines known to be produced by prostate cancer cells, TGFβ and clusterin have previously been reported to possess anti-apoptotic activity (Teicher et al., 1997).

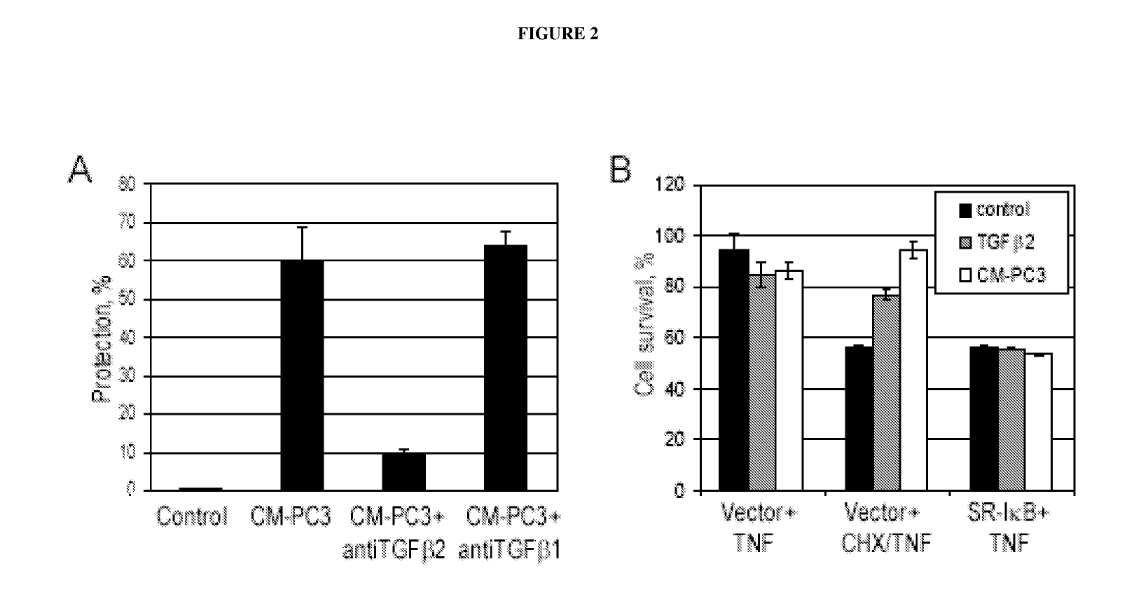

In order to determine whether TGFβ was responsible for the inhibition of TNF-mediated apoptosis, protection experiments were performed as described in Example 2 using conditioned media from PC3 cells together with neutralizing polyclonal antibodies against TGFβ1, TGFβ2, or clusterin. Anti-clusterin (data not shown) and anti-TGFβ1 (FIG. 2A) failed to reduce the protective effect of PC3-conditioned media. However, neutralizing polyclonal antibodies against TGFβ2 almost completely blocked the inhibition of apoptosis provided by PC3-conditioned media (FIG. 2A). As a confirmation of these results, purified recombinant TGFβ2 consistently mediated a protective effect similar to that of conditioned media from PC3 cells (data not shown)

### EXAMPLE 5

### TGFβ2 Mediates the NF-κB-Activating Effect o9f PC3-Conditioned Media

Based on the results in Example 3 and Example 4, we decided to test whether TGFβ2 in the PC3-conditioned media is also responsible for the activation of NF-κB. We produced cells in which the NF-κB response was suppressed by the IκB super-repressor (SR-IκB, Miagkov et al., 1998). FIG. 2B shows that these cells were sensitive to TNF even in the absence of CHX, which usually prevents the induction of endogenous NF-κB by TNF. The sensitivity of these cells to TNF alone, which is induced by the IκB super-repressor, could not be overcome by pre-incubating the cells with conditioned media from PC3 cells or by recombinant TGFβ2 (FIG. 2B). This demonstrates that TGFβ2 protects indicator cells from TNF-mediated apoptosis through the activation of NF-κB.

### EXAMPLE 6

### Constitutive Activation of NF-κB Correlates with Total TGFβ2

PC3 and many other tumor cell lines with constitutive activation of NF-κB secrete TGFβ2. Since the NF-κB inducing effect of PC3-conditioned media could be neutralized by anti-TGFβ2 antibodies, we tested the following tumor cell lines to test whether the level of constitutive activation of NF-κB in these cell lines correlated to the levels of TGFβ2 in conditioned medium from these cells: human normal fibroblast WI38 cells, human fibrosarcoma HT1080 cells, human glioma T98G cells, human melanoma Mel-29 cells, human prostate cancer DU145 cells (kind gift from Tapas DasGupta, the Department of Surgical Oncology, University of Illinois at Chicago), human prostate cancer LNCaP cells, 293C6 and C6P1Z12. C6P1Z12 is a mutant 293-derived cell line selected for constitutive activation of NF-κB (S. Sathe et al., manuscript submitted). 293C6, C6P1Z12, WI38, HT1080, T98G, DU145, CCL64 (ATCC), and Mel-29 cells were cultured in DMEM with 10% FCS.

Constitutive activation of NF-κB in the different tumor cell lines was measured by electrophoresis mobility gel shift assay (EMSA). The oligomer used for an NF-κB binding site was from Santa Cruz Biotechnology, Inc. (Santa Cruz, Calif.): 5′-AGTTGAGGGGACTTTCCCAGGC-3′, labeled with [γ-³²P]-ATP by the polynucleotide kinase method, following the protocol provided by Promega. The cells were washed and collected in 1× PBS and pelleted at 3,000×g at 4° C. for 4 min. The cells were then lysed in hypotonic buffer [20 mM HEPES, pH 7.9, 20 mM NaF, 10 mM Na₃VO₄, 2 mM Na₄P₂O₇, 10 mM EDTA, 10 mM EGTA, 20 mM DTT, 100 mM NaCl, 10% (v/v) glycerol, 1 μg leupeptin, 1 μg/ml pepstatin, 1 μg/ml aprotonin, and 0.5 mM phenylmethanesulfonyl fluoride (PMSF)]. The mixture was vortexed and kept on ice for 15-20 min. Samples were centrifuged at 15,000×g at 4° C. for 4 min. Equal amounts of supernatant solution (normalized for total protein) were incubated with 5 μg/μl of poly (dI-dC) in binding buffer [20 mM HEPES, pH 7.9, 60 mM KCl, 4 mM MgCl₂, 0.2 mM EDTA, 1 mM DTT, 10% (v/v) glycerol, and 2% (v/v) polyvinyl alcohol] for 10 min, and then incubated with 1 μl of γ-³²P-labeled κB probe for another 20 min at room temperature. Samples were loaded onto 6% polyacrylamide gels (acrylamide: N,N′-methylene bisacrylamide, 30:1) in 0.25× Tris borate buffer, pH 8.0. After electrophoresis, the gels were dried and analyzed by autoradiography at −80° C.

The level of TGFβ in the cell-conditioned medium was determined by ELISA. Conditioned media was collected as described above. The Quantikine-Human TGFβ2 Immunoassay was carried out following the protocol from R&D Systems. The monoclonal antibody used in the Quantikine-Human TGFβ2 Immunoassay is specific for the active form of TGFβ2. Total TGFβ2 was measured by first adding 25 μl of 1N HCl to 125 μl samples in order to convert latent TGFβ2 into active TGFβ2. Active TGFβ2 was measures by not performing the activation step. Latent TGFβ2 was measured by taking the difference of total and active TGFβ2 levels. The amount of TGFβ2 was normalized to the cell number, determined in parallel. Neutralizing anti-TGFβ2 antibody (R&D Systems) was used to test the specificity of the ELISA assay.

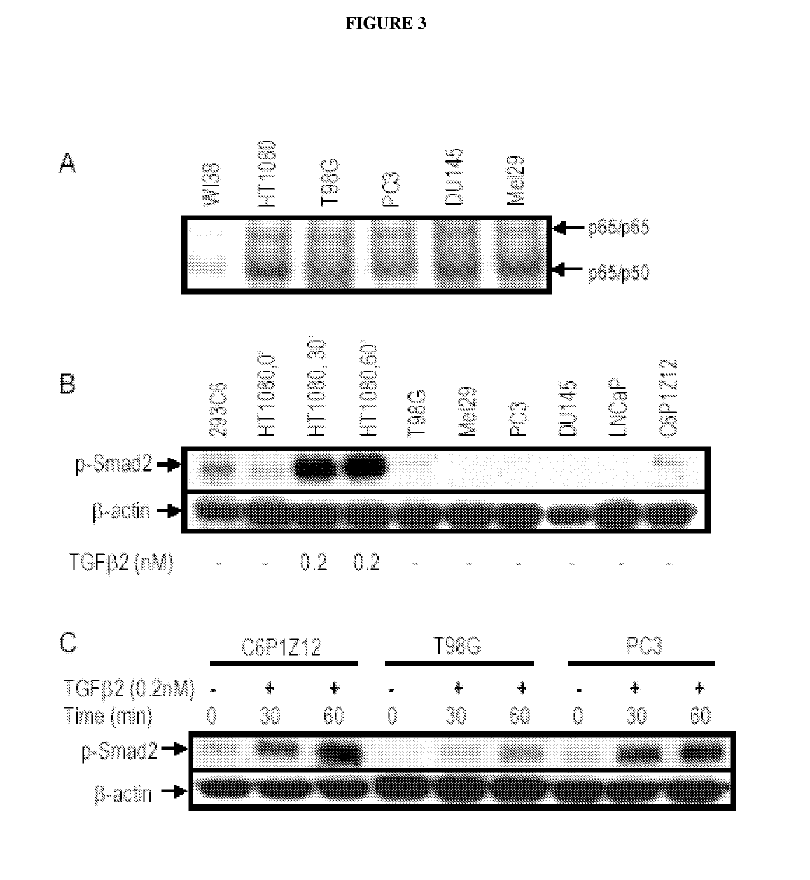

Constitutive activation of NF-κB in some of the tumor cell lines is shown by EMSA is shown in FIG. 3A. Table 1 and FIG. 3A demonstrate that constitutive activation of NF-κB in each of the test tumor cell lines correlates well with the secretion of TGFβ2 (active+inactive). Interestingly, constitutive activation of NF-κB in the tumor cell lines does not correlate to levels of active TGFβ2. In addition, most of the secreted TGFβ2 in the conditioned medium is present as the latent form, both for the tumor cell lines and for C6P1Z12, a mutant 293-derived cell line selected for constitutive activation of NF-κB.

TABLE 1
Correlation of TGFβ2 levels to Constitutive Activation of NF-κB

| | Total | Active | Latent | |
|---|---|---|---|---|
| Conditioned | TGFβ2 | TGFβ2 | TGFβ2 | Constitutively |
| Media | (pg/10⁶) | (pg/10⁶) | (pg/10⁶) | Active NF-κB |
| No cells | 29 | 10 | 19 | − |
| WI38 | 85 | 0 | 85 | − |
| HT1080 | 370 | 0 | 370 | + |
| T98G | 1700 | 48 | 1650 | + |
| Mel29 | 510 | 0 | 510 | + |
| PC3 | 10,000 | 450 | 9550 | + |
| DU145 | 920 | 27 | 893 | + |
| LNCaP | 100 | 0 | 100 | − |
| 293C6 | 68 | 0 | 68 | − |
| C6P1Z12 | 8500 | 200 | 8300 | + |

We also tested each of the cell lines for constitutive activation of Smad2. Western analysis was performed on each of the conditioned cell mediums to determine whether Smad2 was activated by phosphorylation. Each of the cell lines were cultured to 90-100% confluency then treated with TGFβ2 for 0, 30 or 60 min. Cells were washed with 1×PBS, and pelleted at 3,000×g at 4° C. for 4 min. Cell pellets were lysed with RIPA buffer (1×PBS, 1% Nonidet P-40, 0.5% sodium deoxycholate, 0.1% sodium dodecyl sulfate (SDS), 1 mM PMSF, 10 μg/ml aprotinin, 5 μg/ml leupeptin, 10 μg/ml pepstatin, 1 mM Na₃VO₄). Cellular debris was removed by centrifugation at 16,000×g for 10 min. The amount of protein in the supernatant solution was determined and samples were heat-treated in 2×SDS sample loading buffer (20% glycerol, 10% β-mercaptoethanol, 6% SDS, 25 mM Tris-HCl, pH 6.7, and 0.2 mg/ml bromophenol blue) at 100° C. for 5 min. Equal amounts of samples were loaded, fractioned directly by SDS-polyacrylamide gel electrophoresis and transferred to nitrocellulose membranes. Immunoblot analysis was performed with the following primary antibodies: rabbit polyclonal anti-phospho-(Ser465/467)Smad2 (Upstate Biotechnology, Lake Placid, N.J.), or mouse monoclonal anti-β-actin (Labvision Corporation, Fremont, Calif.) at 1:1,500 dilution into 5% milk in 1×TBST (10 mM Tris-HCl, pH 8.0, 150 mM NaCl, 0.1% Triton) at room temperature for 1 h. Hybridization was visualized with horseradish peroxidase-coupled secondary antibodies using the ECL Western blotting detection system (Perkin Elmer Life Sciences).

Consistently, none of the cells examined showed constitutive activation of Smad2 (FIG. 3B). However, the Smad pathway was capable of responding to active TGFβ2 in these cells, as judged by Western analysis (FIGS. 3B and 3C).

### EXAMPLE 7

### Both Active and Latent TGFβ can Activate NF-κB

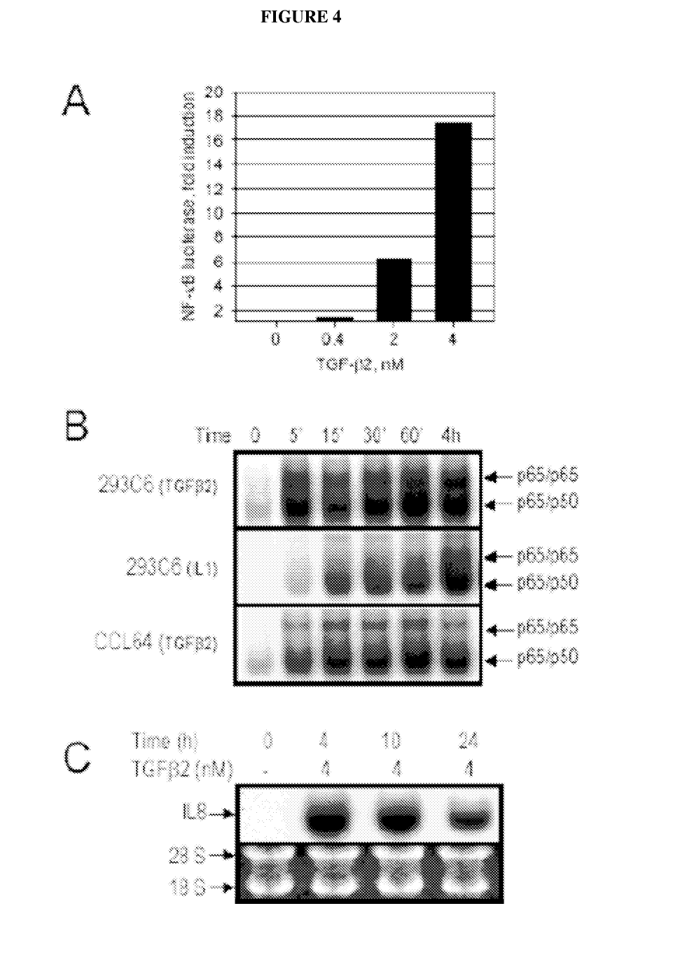

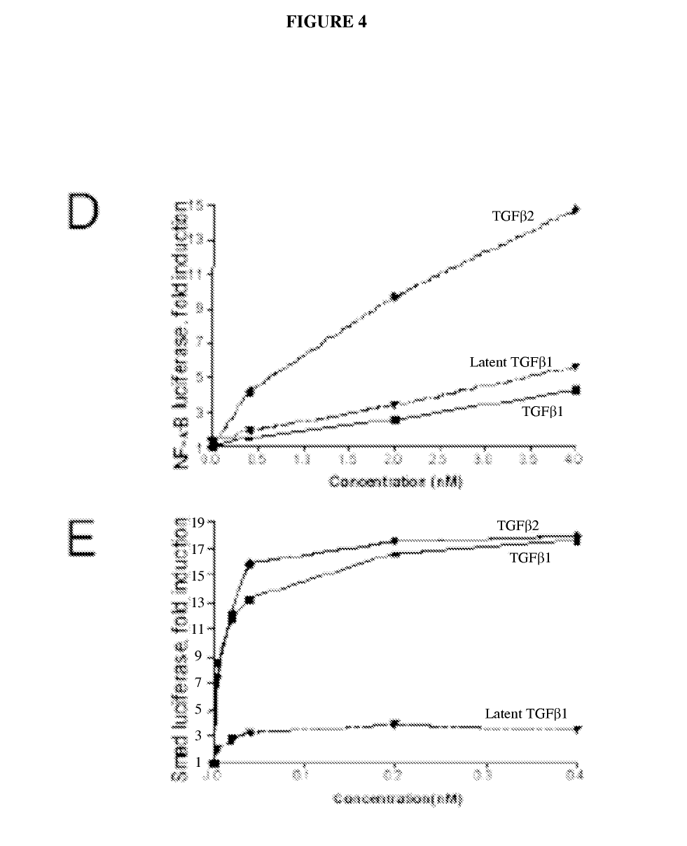

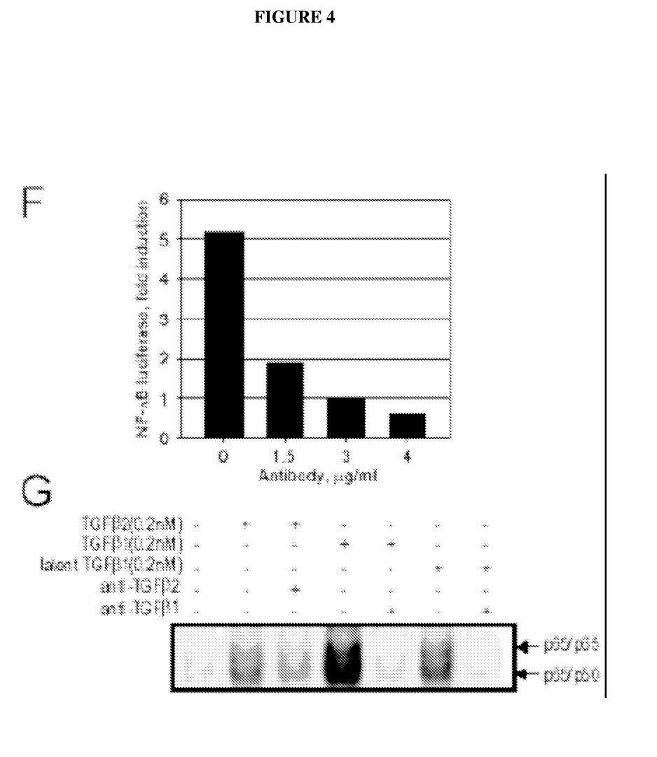

The results in Example 6 suggest that both the latent and active forms of TGFβ2 are capable of activating NF-κB. In order to more directly whether the latent form of TGFβ2 is able to activate NF-κB, we established indicator cells by stably transfecting 293 cells with a κB-luciferase construct. To establish the stable 2931L1R NF-κB indicator cells, a pBabe puromycin resistance plasmid was co-transfected with an NF-κB reporter construct. All transfections were carried out using the Lipofectamine Plus reagent (Invitrogen Life Technologies, Carlsbad, Calif.). Efficiencies of transient transfections were normalized by determining β-galactosidase activity in cells co-transfected with a pCMVLacZ β-galactosidase reporter plasmid. Relative luminescence was normalized to total protein, assayed with the Bio-Rad Protein Assay reagent (Bio-Rad Laboratories, Hercules, Calif.). When the 293 indicator cells were treated with purified recombinant TGFβ2, NF-κB was activated in a dose-dependent manner (FIG. 4A). EMSA showed that the activation occurred within 5 min and persisted for at least 4 h (FIG. 4B).

The activation of IL8, a typical NF-κB target gene, was measure by Northern analysis. A human IL8 cDNA fragment was labeled with [α-³²P]-dCTP by using the Megaprime DNA labeling system, following the protocol provided by Amersham Biosciences (Buckinghamshire, England). The indicator cells were cultured to ˜90% confluent were treated with TGFβ2 (4 nM) for 4 or 10 h, then washed with cold 1×PBS. Total RNA was extracted with the TRIzol reagent at room temperature, following the protocol provided by Invitrogen Life Technologies (Carlsbad, Calif.). Fifteen μg of total RNA was loaded into each lane, electrophoresed in an agarose-formaldehyde gel and transferred onto a Hybond-N⁺ membrane (Amersham Pharmacia Biotech, Piscataway, N.J.). After UV cross-linking, the transfers were hybridized with [α-³²P]-dCTP-labeled probes and analyzed by autoradiography at −80° C. FIG. 4C indicates that transcription of IL8 was induced by treatment with TGFβ.

Since maximal activation of NF-κB or Smads occurred at different doses of TGFβ2, we believed that the two responses to TGFβ2 may be due to the activation of distinct signaling pathways. We tested both active and latent TGFβ1 in 293 indicator cells and found that either could activate NF-κB (FIG. 4D). Active TGFβ2 was much more potent than active or latent TGFβ1 in this assay (FIG. 4D). Active TGFβ1 and β2 had similar effects on the activation of Smads and, as expected, latent TGFβ1 had no effect on Smad activation (FIG. 4E).

The activation of NF-κB by latent TGFβ1 is specific, since a polyclonal antibody against TGFβ1 blocked activation in a dose-dependent manner in 293C6 cells (FIG. 4F). In normal WI38 fibroblasts, the same antibody blocked the activation of NF-κB by either active or latent TGFβ1 (FIG. 4G), showing clearly that these activations are not caused by impurities in the preparations. Therefore, the anti-TNF, NF-κB-inducing effects of media conditioned by tumor cells that exhibit constitutively active NF-κB is determined by the production of the latent form of TGFβ2, which is also incapable of activating Smad-dependent signaling.

### EXAMPLE 8

### The Viability of PC3 Cells Depends on TGFβ

We analyzed the effect of latent TGFβ2 on the phenotype of PC3 cells by suppressing its expression with a small interfering RNA (siRNA). To construct a siRNA-TGFβ2 vector, a nucleotide cassette containing an inverted repeat of the target sequence GAAATGTGCAGGATAATTG, homologous to the 932-950 region of human TGFβ2 cDNA, spaced by the 9-nucleotide sequence TTCAAGAGA and a polyT stretch as a stop codon for RNA polymerase III, was cloned under control of the H1 promoter (Myslinski et al., 2001) and inserted into the 3′LTR of the retroviral vector pLPCX (Miller and Rosman, 1989). Colonies were counted 10 days after puromycin selection. Mixed populations of transfected cells were propagated and tested for TGFβ2 secretion.

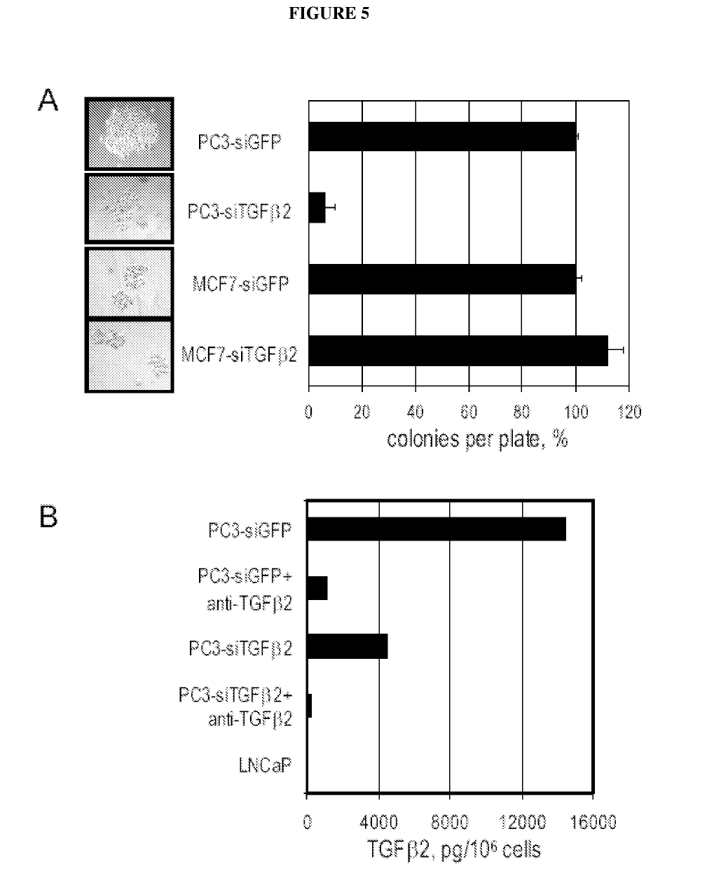

PC3 cells were infected with the retrovirus (pLPCX-siTGFβ2). A construct expressing siRNA against GFP was used as a control. MCF7 and LNCaP cells, infected with the same virus, were used as examples of cells that do not produce TGFβ2. Interestingly, the number and sizes of colonies from pLPCX-siTGFβ2-infected PC3 cells was dramatically reduced in comparison to cells infected with the control virus, whereas the siTGFβ2 RNA had no effect on MCF7 (FIG. 5A) or LNCaP (data not shown) cells.

Rare, slowly growing colonies, formed after transduction with anti-TGFβ2 siRNA, were expanded and tested for TGFβ2 production in comparison with similar colonies generated after transfection with control siRNA (FIG. 5B). Conditioned media from PC3 cells transduced with siRNA against TGFβ2 contained four times less total TGFβ2 (latent plus active) than did media from PC3 cells transduced with siRNA against GFP, determined by ELISA assay, and the majority of the TGF-β2 was in the latent form. The former media were proportionally less capable of inducing NF-κB in indicator cells (data not shown). TGFβ2 production gradually increased during propagation of the siTGFβ2-PC3 cell population, up to the level of the original PC3 cells, suggesting that TGFβ2 provides a selective advantage (data not shown).

### EXAMPLE 9

### Production of Uncleavable Variant of TGFβ2

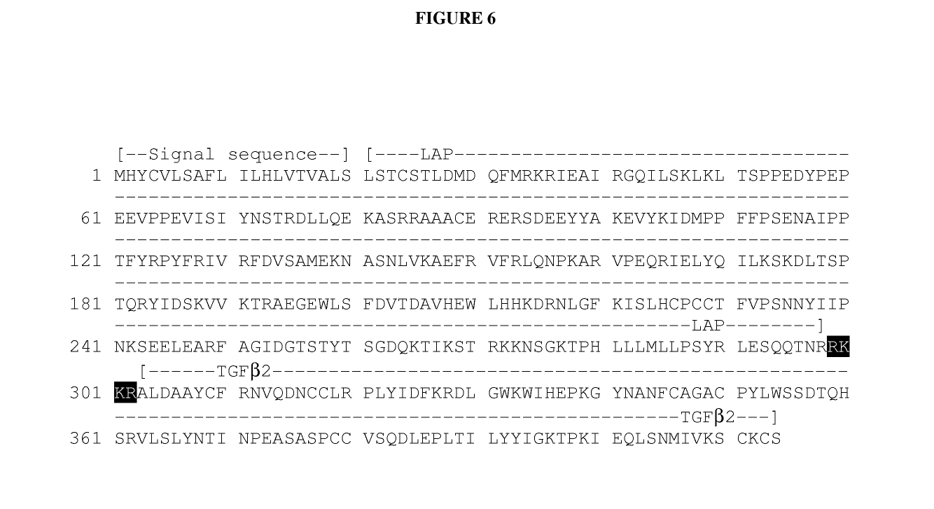

TGFβ2 is synthesized as a precursor protein of either 414 (FIG. 6) or 442 amino acids. The 29 amino acid insertion replaces Asn116 in the 414 amino acid precursor as a result of alternative splicing. Proteolytic processing in two sites (between Leu19 and Ser20 and between Arg302 and Ala303) takes place in the Golgi apparatus prior to secretion, giving rise to a non-covalently bound complex of two homodimers: LAP (amino acids 20-302) and mature TGFβ2 (amino acids 303-414) (in the case of the 414 amino acid precursor). The 1-19 peptide is considered to be the Golgi translocation signal.

In the majority of cell types, latent TGFβ is secreted as a complex with latent TGFβ binding protein (LTBP), called large latent TGFβ complex. On LTBP-null background, TGFβ mostly remains in the Golgi in an unprocessed status, because its secretion is impeded and its proper folding is compromised. However, in some cells (especially in tumors), LTBP is not necessary for successful secretion of the small latent TGFβ complex. We have already demonstrated that in our cell system, LTBP is not necessary for TGFβ2 secretion.

TGFβ1 and 2 are similarly processed by furin, a Golgi resident protease of the proprotein convertase family. The consensus site for furin is R-X-R/K-R. Accordingly, substitution of the last Arg of the cleavage site consensus with Ser, as well as other mutational substitutions, are expected to give rise to an uncleavable form of TGFβ2 precursor protein as it has shown for other proteins processed via furin-mediated cleavage.

The cDNA of precursor TGFβ2 is obtained from prostate cancer PC3 cells by RT-PCR. TGFβ2 precursor cDNA are modified by site-directed mutagenesis to generate furin uncleavable forms. Specifically, these mutations include: R302→S or double mutation of both consensus arginines—R302→S, R299→S.

Rationally designed truncated versions of TGFβ2 are also constructed using standard PCR-based approaches. The cDNA of wild type, mutant uL-TGFβ2 precursors and LAP are cloned into a lentiviral vector and delivered to either human LNCaP, Hela, WI-38 cells or mouse NIH-3T3 fibroblasts that express only minute amounts of endogenous TGFβ2 but produce high levels of recombinant TGFβ2 upon transduction with the lentivirus bearing full length wild type TGFβ2 cDNA.

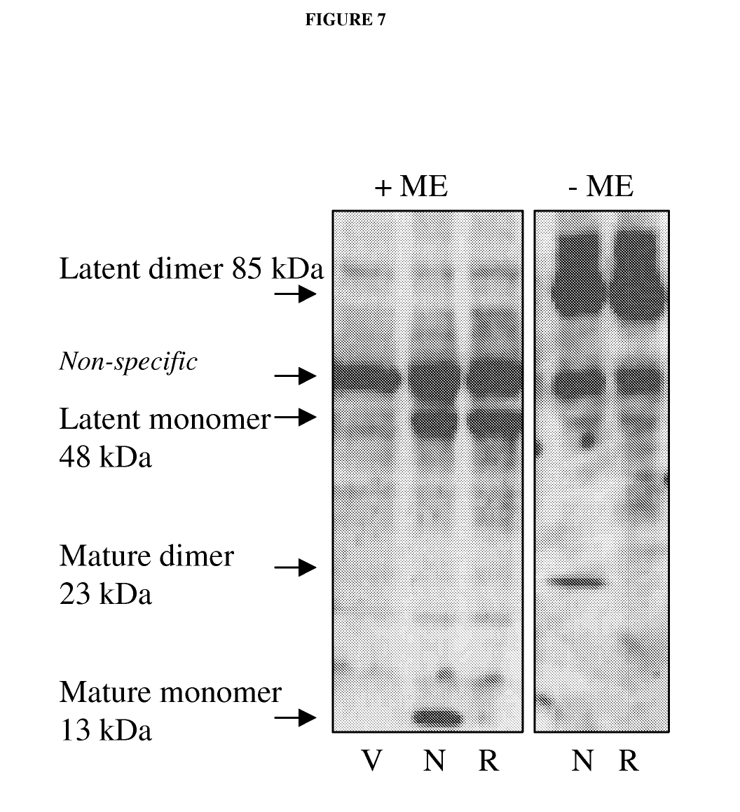

We generated lentivirus expression construct for wild type precursor TGFβ2 and a mutated form containing the substitution of Arg for Ser in the furin cleavage site. For controls, we created vector with LAP portion of TGFβ2 or used empty vector. WI-38, Hela and LNCaP cells were transduced with the plasmids or control empty vector and analyzed for the expression of TGFβ2 by Western blotting using polyclonal anti-TGFβ2 antibodies. FIG. 7 demonstrates the production of TGFβ2 by LNCaP cells. TGFβ can be processed into the mature form by thermal treatment of its latent form at 80-100° C. One set of LNCaP cell lysates was incubated at 90° C. for 5 min in buffer containing B-mercaptoethanol to activate latent TGFβ2 and to compare the effect of such treatment on normal and mutant forms of TGFβ2. The results demonstrated that control LNCaP cells do not express any TGFβ2 (line V) while the transduced cells express rather high levels of both generated forms of TGFβ2: wild type (line N) and mutant (line R). Most importantly, only wild type latent TGFβ2 (48 kDa monomer) was processed into mature TGFβ2 (13 kDa monomer) by B-mercaptoethanol in combination with thermal treatment. Therefore, the mutation inserted into the furin cleavage site was able to prevent the activation of TGFβ2.

### EXAMPLE 10

### Uncleavable Latent TGFβ Activates NF-κB

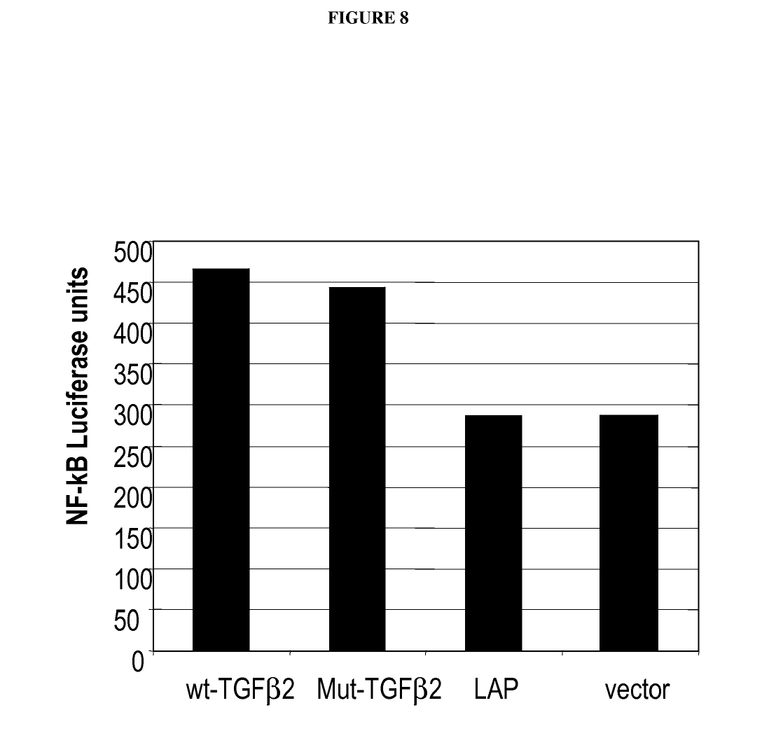

In order to determine whether the uncleavable mutant form of TGFβ2 is able to induce NF-κB, we used H1299 (human lung carcinoma epithelial cells) reporter cells stably carrying an NF-κB-responsive luciferase reporter construct containing three NF-κB-binding sites from E selectin promoter combined with Hsp70 minimal promoter. The H1299 NF-κB reporter cell line was treated with conditioned media from WI-38 (FIG. 8), LNCaP or Hela cells transduced with either wild type precursor TGFβ2 cDNA, mutant uncleavable TGFβ2 precursor cDNA, or control empty vector or LAP portion of TGFβ2. Luciferase activity was measured in cell lysates six hours after addition of TGFβ2 into the medium. Both types of conditioned media from WI-38 cells transduced with wild type or uncleavable forms of TGFβ2 were able to activate NF-κB transactivation in contrast to the control conditioned media from empty vector transduced cells.

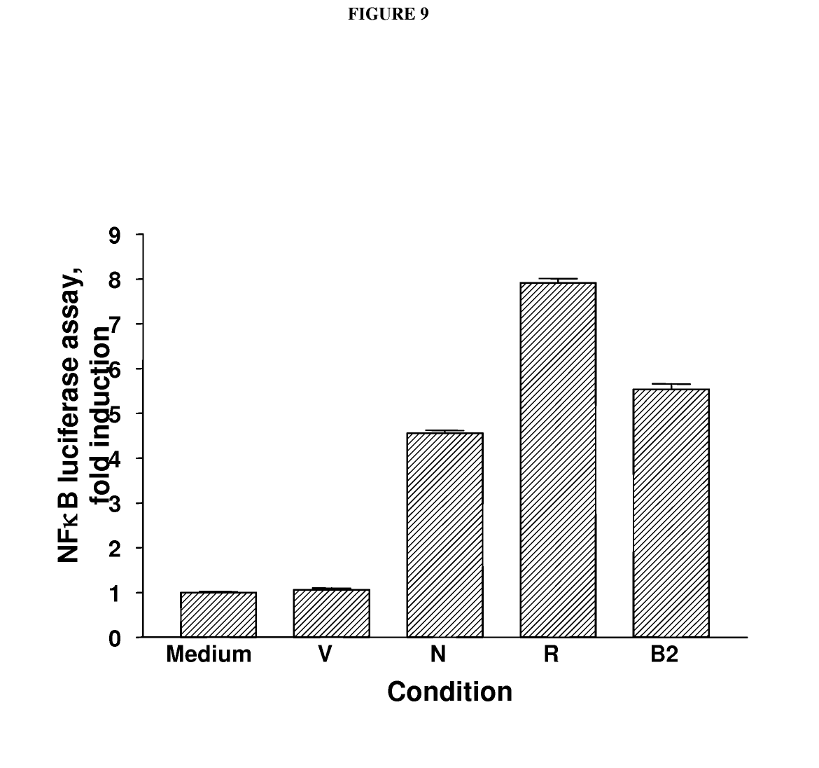

In order to determine whether latentTGFβ2 is able to induce NF-κB in TGFβ2 producing cells, we used 293 (human kidney embryo cells) reporter cells stably carrying an NF-κB-responsive luciferase reporter gene. The reporter construct contains three NF-κB-binding sites from E-selectin promoter combined with Hsp70 minimal promoter and is routinely used for the detection of NF-κB status of cells. The 293 NF-κB reporter cell line was transfected with wild type precursor TGFβ2 cDNA, mutant uncleavable TGFβ2 precursor cDNA, or control empty vector and co-transfected with β-galactosidase as the transfection efficiency control. Luciferase activity was measured in cell lysates 48 hours after transfection. Recombinant TGFβ2 (1 ng/ml) was used as a positive control, and the data was normalized to the β-galactosidase reading. The results of NF-κB activation in FIG. 9 demonstrate the capacity of both types of TGFβ2, wild type and uncleavable forms, to activate NF-κB transactivation, in contrast to the cells transfected with empty vector.

### EXAMPLE 11

### Uncleavable Form of TGFβ2 cannot be processed into Active TGFβ2

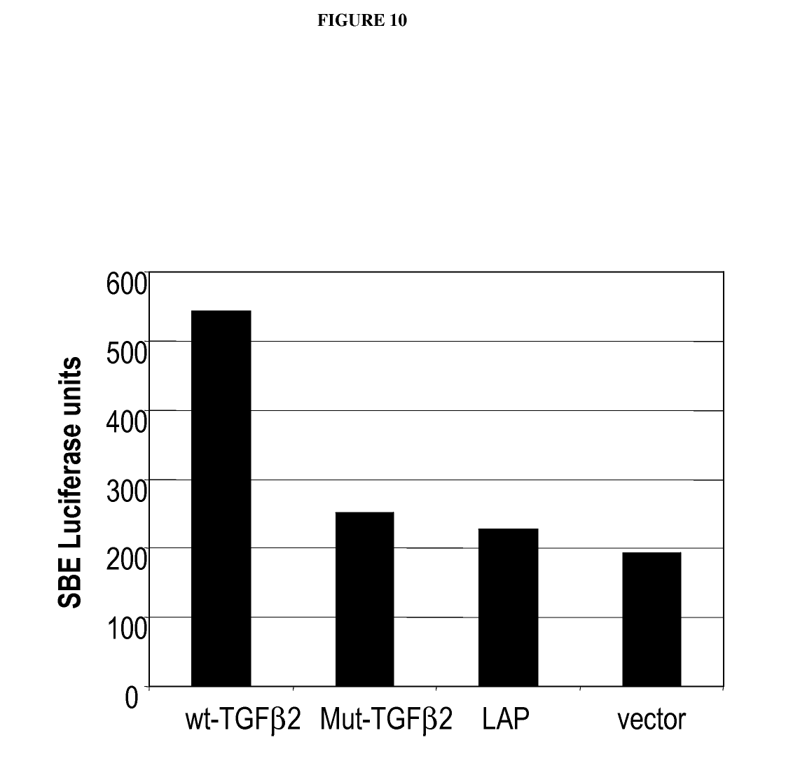

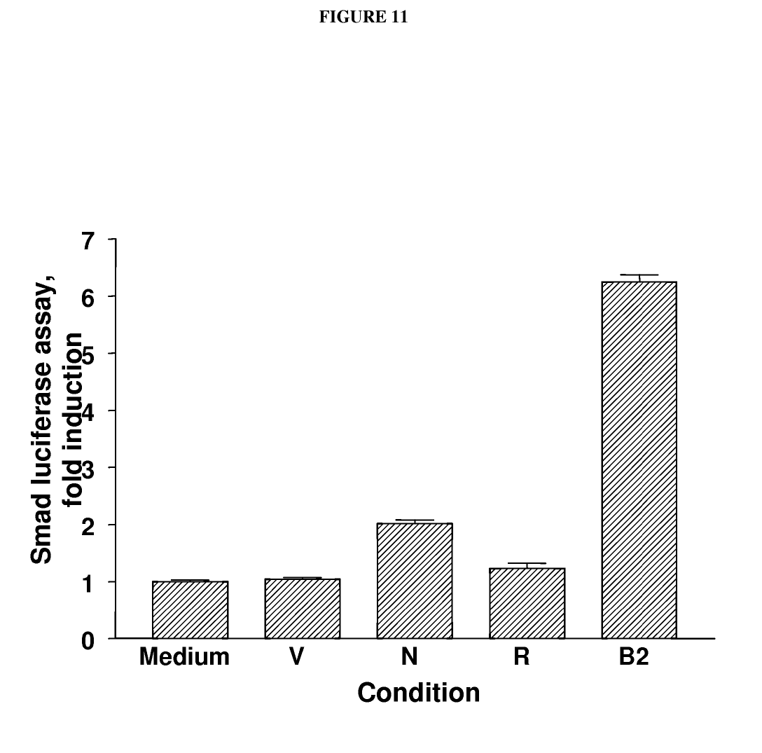

Since it has been shown that latent TGFβ2 can be processed to active TGFβ2 by thermal (100° C.) or acid (pH 4.1-3.1) treatment, we incubated conditioned medium from LNCaP cells transduced with different forms of TGFβ2 at 100° C. for 5 min (FIG. 10) or in HCl and perform a Smad2 luciferase reporter assay using transiently transfected mouse fibroblast NIH-3T3 cells. We observed the only activation of Smad2 signaling in the luciferase reporter cells by wild type TGFβ2 containing media, not mutant or any of control conditioned media.

Smad2 luciferase reporter assay was also performed with 293 reporter cells stably carrying a Smad2-responsive luciferase reporter gene and transfected with wild type precursor TGFβ2 cDNA, mutant uncleavable TGFβ2 precursor cDNA, or control empty vector and co-transfected with β-galactosidase as the transfection efficiency control. Luciferase activity was measured in cell lysates 48 hours after transfection. Recombinant TGFβ2 (1 ng/ml) was used as a positive control and the data was normalized to the β-galactosidase reading. Only recombinant TGFβ2 and, to a much lesser extent, wild type TGFβ2 activated Smad2 signaling in the luciferase reporter cells. Mutant TGFβ2 did not activate Smad signaling in 293 indicator cells. Thus, we demonstrated the effect of NF-κB activation by both forms of TGFβ2, cleavable and uncleavable, while only wild type TGFβ2 was able to activate Smad2 signaling in luciferase reporter assays.

### EXAMPLE 12

### Comparison of TGFβ2 Producing Cells

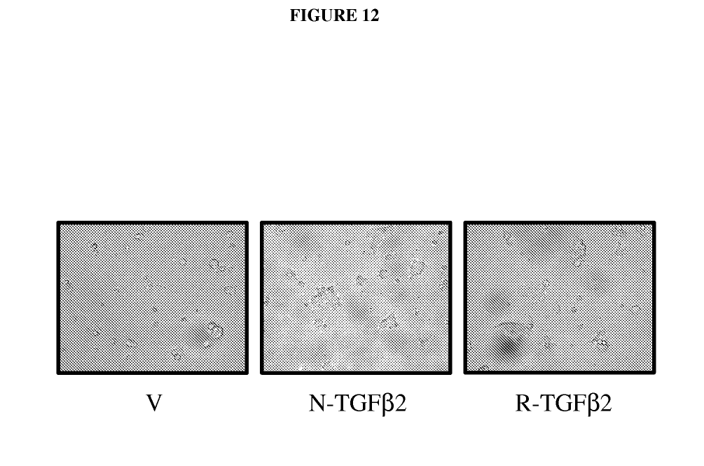

We produced 293 cells permanently producing normal (N) or mutant (R) TGFβ2 by lenti-viral transduction and bleomycin selection. Control cells were transduced with empty vector (V) and also selected with bleomycin. It was noticed during the bleomycin selection that the cells with TGFβ2 grew faster then 293 transduced with vector. To confirm the observation of the beneficial effect of TGFβ2 on cell growth, we put equal amounts of the cells in wells with low cell density and checked the condition of cell cultures in microscope. As shown in FIG. 12, TGFβ2 (both, normal and mutant forms) producing cells demonstrated better adherence and bigger colonies than TGFβ2 non-producing cells, which confirms that uncleavable latent TGFβ2 has growth stimulating activity.

### EXAMPLE 13

### Uncleavable Latent TGFβ2 is a Radioprotectant

Based on the analysis of TGFβ2 production, either LNCaP, Hela or NIH-3T3 cells will be used for the production of uncleavable latent TGFβ2 (uL-TGFβ2). Bulk stocks of uL-TGFβ2 and wild type TGFβ2 (wt-TGFβ2) will be obtained by concentration of the conditioned serum-free phenol red-free medium from the producing cells using a 10 kDa Millipore filter. Control medium will be collected from cells transduced with empty vector and prepared as described above. The control concentrated medium will be tested for the absence of NF-κB activation in vitro and the absence of toxicity in vivo. The concentration of uL-TGFβ2 will be quantitatively determined by ELISA using polyclonal antibodies for Western blotting protein analysis.

The maximal tolerable dose will be determined by the administration of increasing doses of wt-TGFβ2 or uL-TGFβ2 to mice and then monitoring for associated body weight loss and morbidity. Possible systemic inflammatory effects of the different TGFβ2 forms will be assessed in mice by a post-injection measurement of pro-inflammatory cytokine levels and body temperature taken within 3-6 hours interval post-injection. TGFβ2 activates NF-κB, which is known to induce cytokine production. Different cytokines, such as IL-1β, IL-6 and TNFα are largely responsible for the systemic effects of inflammation, including fever, cachexia and hypoglycemia. The levels of one of the most sensitive cytokines, IL-8, will be determined in blood serum using commercially available ELISA kits. Serum will be collected from mice for cytokine concentration measurement within 2 hours and 4 hours after injection. Pro-inflammatory cytokine levels have been shown to peak within this time frame for mice treated with Escherichia coli lipopolysaccharide (LPS), an efficient inductor of both NF-κB and inflammatory response.

The dose of each form of TGFβ2 will be increased up to the appearance of systemic effects of acute inflammation, such as fever, as well as undreamt coat, hunched posture, diarrhea etc. Highest dose causing any of these effects in 30% of animals will be considered as toxic and further radioprotective effects will be studied starting from the highest dose equal to ½ of toxic dose. We will detect increases in IL-8 concentration in the serum of mice treated with TGFβ2 (indicative of activated NF-κB) before appearance of toxic symptoms. Concentration ranges of wt-TGFβ2 and uL-TGFβ2 between those causing increased IL-8 production and toxicity dose will be considered as the therapeutic interval.

Several dilutions of TGFβ2 will be used containing conditioned media starting from the highest dose equal to ½ of toxic dose determined in toxicity studies. Mice will be injected intravenously 1 h before 10 and 15 Gy of whole-body gamma irradiation (these doses usually lead to 100% lethality). We will determine the optimal dosage by applying whole-body gamma irradiation using Shepherd 4000 Ci Cesium 137 source at a dose rate of 2.5 Gy per minute and monitoring the percent of survivors up to 30 days.

Several routes of application of TGFβ2 containing conditioned medium will be tested: intravenous, intraperitoneal, subcutaneous, gavage and its combinations. NIH-Swiss mice, purchased from Harlan, will be injected with different forms of TGFβ2 one hour before whole-body γ-irradiation with 10 Gy for protection from hematopoietic syndrome and 15 Gy for protection from gastrointestinal syndrome using Shepherd 4000 Ci Cesium 137 source at a dose rate of 2.5 Gy per minute. We also plan to administer TGFβ2 by subcutaneous injection of γ-irradiated syngenic NIH-3T3 fibroblasts, producing TGFβ2, 8-24 h before irradiation. Groups of 10-14 animals unified by age and sex will be used in each experiment to achieve statistically significant values monitoring the percent of survivors up to 30 days.

LD₅₀ values will be calculated by performing the irradiation experiments using a dose range of irradiation between 5-15 Gy that usually leads to 10-100% mortality. Dose causing death of 50% of animals at day 7 and day 30 after irradiation will be defined as LD_{50/7} and LD_{50/30} respectively. DMF (dose modification factor, also known as dose reduction factor, DRF) is calculated as a ratio of LD₅₀ of the group of mice treated with TGFβ2 containing medium to LD₅₀ of mice treated with a control medium for a particular survival time point (7- and 30-day survival). The results will show that uL-TGFβ and wild type TGFβ, similar to the NF-κB inducer flagellin.

1. A modified TGFβ polypeptide, wherein the modification reduces the rate of conversion to active TGFβ.

2. The polypeptide of claim 1 wherein the polypeptide comprises SEQ ID NO:1, and wherein R299 or R302, or a combination thereof, is substituted with serine.

3. A pharmaceutical composition comprising the polypeptide of claim 1.

4. A method of protecting a patient from a condition that triggers apoptosis comprising administering to a patient in need thereof a composition comprising a pharmaceutically effective amount of latent TGFβ.

5. The method of claim 1 wherein latent TGFβ is administered prior to, together with, or after a treatment that triggers apoptosis.

6. The method of claim 5, wherein the treatment is a cancer treatment.

7. The method of claim 5, wherein the treatment is chemotherapy or radiation therapy.

8. The method of claim 4, wherein the condition is a stress selected from the group consisting of radiation, wounding, poisoning, infection and temperature shock.

9. The method of claim 4, wherein the latent TGFβ is a polypeptide according to claim 1.

10. A method of diagnosis of a cancer in a mammal comprising:
(a) incubating a sample obtained from said mammal with an agent which specifically detects the presence of latent TGFβ; and
(b) comparing the level of latent TGFβ to a reference,
whereby a difference in the level of latent TGFβ compared to the reference is indicative of a cancer.

11. A method of screening for a modulator of NF-κB comprising:
(a) adding a suspected modulator and TGFβ to an NF-κB activated expression system;
(b) separately adding TGFβ to an NF-κB activated expression system; and
(c) comparing the level of NF-κB activated expression in (a) and (b),
whereby a difference in the level of NF-κB activated expression in (a) and (b) is indicative of a modulator of NF-κB.

12. The method of claim 11 wherein the TGFβ is latent TGFβ.

13. A method of screening for a modulator of TGFβ comprising:
(a) adding a suspected modulator and TGFβ to a TGFβ activated expression system;
(b) separately adding TGFβ to a TGFβ activated expression system; and
(c) comparing the level of TGFβ activated expression in (a) and (b),
whereby a difference in the level of TGFβ activated expression in (a) and (b) is indicative of a modulator of TGFβ.

14. The method of claim 13 wherein the TGFβ is latent TGFβ.
